(19) United States
(12) Patent Application Publication
(10) Pub. No.: US 2026/0108780 A1
(43) Pub. Date: Apr. 23, 2026

(54) SYSTEM AND METHOD FOR CONDUCTING TRAINING SESSIONS

(71) Applicant: VIXON LLC, PEMBROKE PINES, FL (US)

(72) Inventors: Agostina GALIMBERTI, Port Saint Lucie, FL (US); Paulo Suarez PEREZ, Port Saint Lucie, FL (US)

(21) Appl. No.: 19/348,955

(22) Filed: Oct. 3, 2025

Related U.S. Application Data
(63) Continuation-in-part of application No. 18/921,249, filed on Oct. 21, 2024, now Pat. No. 12,453,900.

(51) Int. Cl. A63B 24/00; A63B 71/06

(52) U.S. Cl. CPC A63B 24/0075; A63B 71/0622; A63B 2024/0081; A63B 2071/0661

(57) ABSTRACT

A system for conducting a training session for a plurality of players comprises a training device, a hub network device, and wearable devices. The training device displays icons representing players associated with the wearable devices. The training device creates a plurality of virtual teams based on the selection of the players and associates the virtual teams with different colors. In addition, the training device transmits signals to illuminate visual indicators of the wearable devices of the players in each virtual team with respective associated colors to indicate commencement of different predefined activities. Furthermore, the training device also associates at least one icon representing at least one player with a different color from a previously associated color and transmits a signal to at least one wearable device corresponding to the player to illuminate the visual indicator of the wearable device in the different color to indicate commencement of a different predefined activity by the player.

### CROSS REFERENCE TO RELATED APPLICATIONS

The present application is a continuation-in-part of U.S. Non-Provisional application Ser. No. 18/921,249, filed Oct. 21, 2024, entitled “SYSTEM AND METHOD FOR CONDUCTING TRAINING SESSIONS” which is incorporated by reference herein in its entirety.

### BACKGROUND OF THE INVENTION

To encourage team-play and team coordination, coaches often selectively segregate players into different teams and conduct training sessions. However, conducting such training sessions each time can become time consuming, monotonous, and predictable overtime. Moreover, performance, agility, and coordination skills of each individual player cannot improve by conducting similar type of training sessions repeatedly. Thus, it can be desirable for the coaches to make the training sessions more engaging for the players each time. It can also be desirable for the coaches to involve the players in different teams, situations, positions, and/or conditions, to evaluate the performance of the players. Such performance evaluations can also help coaches to better understand team dynamics and strategize game plans based on the team dynamics.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 illustrates an environment employing an exemplary system for conducting a training session for players, in accordance with some embodiments;

FIG. 2 illustrates a schematic block diagram of an exemplary wearable device included in the exemplary system of FIG. 1, in accordance with some embodiments;

FIG. 3 illustrates a schematic block diagram of an exemplary training device included in the exemplary system of FIG. 1, in accordance with some embodiments;

FIG. 4 illustrates an exemplary graphical user interface of the training device of FIG. 3 for registration of an instructor/coach managing or conducting the training session;

FIG. 5 illustrates an exemplary graphical user interface of the training device of FIG. 3 for establishing connection and/or communication with the wearable devices of players included in the exemplary system of FIG. 1, in accordance with some embodiments;

FIG. 6 illustrates an exemplary graphical user interface of the training device of FIG. 3 for identifying wearable devices of players paired with the training device of FIG. 3, in accordance with some embodiments;

FIGS. 7 and 8 illustrate an exemplary graphical user interface of the training device of FIG. 3 for creating virtual teams of players with corresponding wearable devices, in accordance with some embodiments;

FIG. 9 illustrates an exemplary graphical user interface of the training device of FIG. 3 for conducting a game between two virtual teams, in accordance with some embodiments;

FIG. 10 illustrates an exemplary graphical user interface of the training device of FIG. 3 for conducting a game between two virtual teams with double agents, in accordance with some embodiments;

FIG. 11 illustrates an exemplary graphical user interface of the training device of FIG. 3 for conducting a game between two un-balanced virtual teams, in accordance with some embodiments;

FIG. 12 illustrates an exemplary graphical user interface of the training device of FIG. 3 for conducting an exercise session for players, in accordance with some embodiments;

FIG. 13 illustrates an exemplary graphical user interface of the training device of FIG. 3 for conducting a game between two virtual teams, in accordance with some additional embodiments;

FIG. 14 illustrates a schematic block diagram of a server included in the exemplary system of FIG. 1, in accordance with some embodiments; and

FIGS. 15A through 15B is a flowchart of an exemplary method for conducting a training session for a plurality of players using the training device of FIG. 3, in accordance with some embodiments.

Skilled artisans will appreciate that elements in the figures are illustrated for simplicity and clarity and have not necessarily been drawn to scale. For example, the dimensions of some of the elements in the figures can be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION OF THE INVENTION

In one aspect, a system for conducting a training session for a plurality of players includes a plurality of wearable devices correspondingly associated with the players and a training device associated with an instructor and in communication with the wearable devices. Each wearable device includes a visual indicator capable of being illuminated in a plurality of distinct colors. The training device is configured to display, via a user interface, a plurality of icons correspondingly representing the plurality of players associated with the wearable devices upon establishment of the communication between the plurality of wearable devices and the training device. The training device is also configured to automatically create, via a processor of the training device and at least one artificial intelligence model implemented by the processor, a set of randomized virtual teams of the plurality of players on the user interface based on a count of the plurality of wearable devices in communication with the training device. The training device is also configured to create, via a processor of the training device, a first virtual team of the created set of randomized virtual teams by selecting a first subset of icons of the plurality of icons corresponding to a first subset of players of the plurality of players. The training device is also configured to create a second virtual team of the create set of randomized virtual teams by selecting a second subset of icons of the plurality of icons corresponding to a second subset of players of the plurality of players. The first subset of players is different from the second subset of players. Further, the training device is also configured to automatically generate, via the processor and the at least one artificial intelligence model, a plurality of predefined activities to be performed by the created set of randomized virtual teams at a plurality of time intervals respectively. Further, the training device is configured to automatically commence, via the processor and the at least one artificial intelligence model, a first predefined activity of the plurality of predefined activities for a first time interval of the plurality of time intervals.

The training device is further configured to associate, via the processor, the first virtual team and the first subset of icons with a first color of the plurality of distinct colors by selecting the first color, and the second virtual team and the second subset of icons with the second color of the plurality of distinct colors by selecting the second color to commence the first predefined activity. In addition, the training device is configured to illuminate, via the processor and a transceiver of the training device, the visual indicator of each of the first subset of wearable devices associated with the first subset of players in the first color and the visual indicator of each of the second subset of wearable devices associated with the second subset of players with the second color. The illumination of the visual indicator in the first color and the second color is indicative of the commencement of a first predefined activity between the first subset of players and the second subset of players. Further, the training device is configured to commence, via the processor and the at least one artificial intelligence model, at least one subsequent predefined activity of the plurality of predefined activities that is different from the first predefined activity for a subsequent time interval of the plurality of time intervals after or during the first time interval. Furthermore, the training device is configured to associate, via the processor, at least one icon of the plurality of icons corresponding to at least one player with at least one color of the plurality of distinct colors to commence the at least one subsequent predefined activity. The training device is also configured to illuminate the visual indicator of the wearable device in the at least one associated color. The illumination of the visual indicator in the at least one color is indicative of the commencement of at least one subsequent predefined activity by the at least one player.

In another aspect, a method for conducting a training session for a plurality of players includes displaying, via a user interface of a training device, a plurality of icons correspondingly representing the players associated with a plurality of wearable devices in communication with the training device. Each wearable device of the plurality of wearable devices includes a visual indicator capable of being illuminated in a plurality of distinct colors. The method also includes creating, via a processor of the training device and at least one artificial intelligence model implemented by the processor, a set of randomized virtual teams of the plurality of players on the user interface based on a count of the plurality of wearable devices in communication with the training device. The method also includes creating, via a processor of the training device, a first virtual team of the created set of randomized virtual teams by selecting a first subset of icons of the plurality of icons corresponding to a first subset of players of the plurality of players, and a second virtual team of the created set of randomized virtual teams by selecting a second subset of icons of the plurality of icons corresponding to a second subset of players of the plurality of players. The first subset of players is different from the second subset of players. The method also includes generating, via the processor and the at least one artificial intelligence model, a plurality of predefined activities to be performed by the created set of randomized virtual teams at a plurality of time intervals respectively. Further, the method includes commencing, via the processor and the at least one artificial intelligence model, a first predefined activity of the plurality of predefined activities for a first time interval of the plurality of time intervals.

The method further includes associating, via the processor, the first virtual team and the first subset of icons with a first color of the plurality of distinct colors, and the second virtual team and the second subset of icons with the second color of the plurality of distinct colors to commence the first predefined activity. The method also includes illuminating, via the processor and a transceiver of the processor, the visual indicator of each of first subset of wearable devices in the first color and the visual indicator of each of the second subset of wearable devices in the second color. The illumination of the visual indicator in the first color and the second color is indicative of the commencement of the first predefined activity between the first subset of players and the second subset of players. Further, the method includes commencing, via the processor and the at least one artificial intelligence model, at least one subsequent predefined activity of the plurality of predefined activities that is different from the first predefined activity for a subsequent time interval of the plurality of time intervals after or during the first time interval. Furthermore, the method includes associating, via the processor, at least one icon of the plurality of icons corresponding to at least one player with at least one color of the plurality of distinct colors to commence the at least one subsequent activity. The method illuminating, via the processor and the transceiver, the visual indicator of the wearable device in the at least one color. The illumination of the visual indicator in the at least one color is indicative of the commencement of at least one subsequent predefined activity by the at least one player.

In yet another aspect, a training device for conducting a training session for a plurality of players includes a transceiver in communication with plurality of wearable devices correspondingly associated with the players. Each wearable device of the plurality of wearable devices includes a visual indicator capable of being illuminated in a plurality of distinct colors. The training device also includes a processor and a memory for storing instructions, that when executed by the processor, causes the processor to display, via a user interface, plurality of icons correspondingly representing the players associated with the wearable devices upon establishment of the communication between the plurality of wearable devices and the training device. The processor is also configured to create, via at least one artificial intelligence model implemented by the processor, a set of randomized virtual teams of the plurality of players on the user interface based on a count of the plurality of wearable devices in communication with the training device. The processor is also configured to create a first virtual team of the created set of randomized virtual teams by selecting a first subset of icons of the plurality of icons corresponding to a first subset of players of the plurality of players. The processor is also configured to create a second virtual team of the created set of randomized virtual teams by selecting a second subset of icons of the plurality of icons corresponding to a second subset of players of the plurality of players. The first subset of players is different from the second subset of players. The processor is further configured to generate, via the at least one artificial intelligence model, a plurality of predefined activities to be performed by the created set of randomized virtual teams at a plurality of time intervals respectively. In addition, the processor is configured to commence, via the at least one artificial intelligence model, a first predefined activity of the plurality of predefined activities for a first time interval of the plurality of time intervals.

The processor is further configured to automatically associate the first virtual team and the first subset of icons with a first color of the plurality of distinct colors, and the second virtual team and the second subset of icons with the second color of the plurality of distinct colors. In addition, the processor is configured to illuminate, via the transceiver, the visual indicator of each of the first subset of wearable devices in the first color and the visual indicator of each of the second subset of icons in the second color. The illumination of the visual indicator in the first color and the second color is indicative of the commencement of the first predefined activity between the first subset of players and the second subset of players. The processor is also configured to commence, via the at least one artificial intelligence model, at least one subsequent predefined activity of the plurality of predefined activities that is different from the first predefined activity for a subsequent time interval of the plurality of time intervals after or during the first time interval. Furthermore, the processor is configured to associate at least one icon of the plurality of icons corresponding to at least one player with at least one color of the plurality of distinct colors to commence the at least one subsequent predefined activity. The processor is also configured to illuminate, via the transceiver, the visual indicator of the wearable device in the at least one color. The illumination of the visual indicator in the at least one color is indicative of the commencement of the at least one subsequent predefined activity by the at least one player.

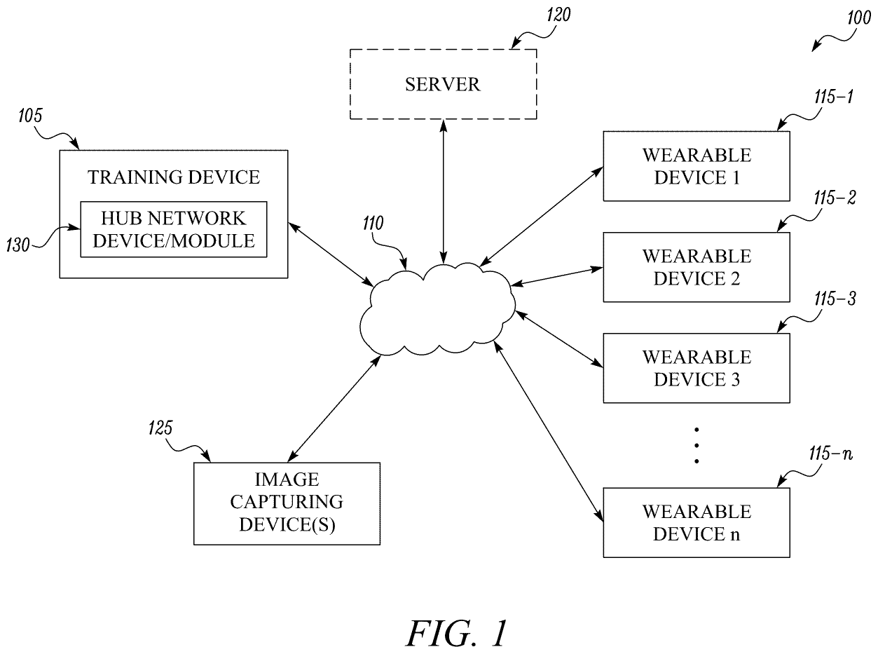

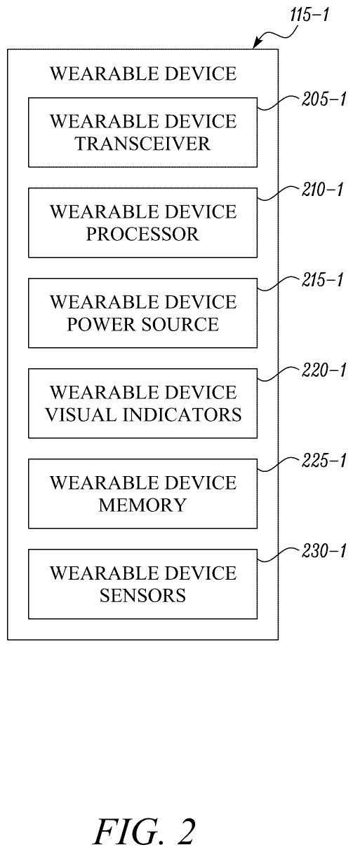

FIG. 1 illustrates an environment employing an exemplary system 100 for conducting a training session for players. The system 100 includes a training device 105 in communication with plurality of wearable devices, for example, 115-1, 115-2, 115-3 . . . 115-n, via a network 110. Examples of the network 110 include, but are not limited to, a Local Area Network (LAN), a Wireless Local Area Network (WLAN), a Wireless Personal Area Network (WPAN) including, but not limited to, a Small Area Network (SAN), and a telecommunication network including, but not limited to, a fourth generation (4G) and a fifth generation (5G) cellular network, and a direct or peer-to-peer communication network such as, but not limited to, a Wi-Fi Direct network and Bluetooth®. In some embodiments, the system 100 also includes a server 120 in communication with the training device 105 and/or the wearable devices, for example, 115-1, 115-2, 115-3 . . . 115-n via the network 110. In some embodiments, the system 100 also includes one or more image capturing devices, 125 herein referred to as ‘image capturing devices(s) 125’, in communication with the training device 105 and/or the server 115 via the network. In some embodiments, the image capturing device(s) 125 is configured to capture the players and/or one or more activities performed by the players. In an exemplary embodiment, the training device 105 is associated with an instructor or a coach managing the training sessions. In an exemplary embodiment, the plurality of wearable devices, for example, 115-1, 115-2, 115-3 . . . 115-n, are associated with the players. It will be appreciated by those of ordinary skill in the art that the training sessions, the instructors, coaches, and/or the players can be associated with any sport including, but not limited to, soccer, football, tennis, and basketball, or any activity including, but not limited to, exercising, and dancing. Examples of the training device 105 include, but are not limited to, computers, laptops, mobile devices, handheld devices, personal digital assistants (PDAs), tablet personal computers, digital notebook, wearables, specialized, customized, and/or purpose-built electronic devices, and any other computing device now known or developed in the future. Examples of the wearable devices, for example, 115-1, 115-2, 115-3 . . . 115-n, include, but are not limited to, sport/smart watches, smart jewelry, smart clothing, fitness tracking devices, head-mounted displays, and implantable devices. In an exemplary embodiment, the wearable devices, for example, 115-1, 115-2, 115-3 . . . 115-n, correspond to smart clothing including, but not limited to, sports vests, jerseys, shirts, T-shirts, shorts, and pants. In accordance with various embodiments, the wearable devices, for example, 115-1 . . . 115-n include one or more wearable device visual indicators, for example, 220-1 . . . 220-n (see FIG. 2), herein referred to as ‘wearable device visual indicator(s)’, respectively capable of being illuminated in a plurality of distinct colors. Examples of the wearable device visual indicator(s), for example, 220-1 . . . 220-n include, but are not limited to, Light Emitting Diodes (LED) lights, digital indicators, electronic visual displays, and/or any other light emitting or display device known now or in future developed.

In some embodiments, the training device 105 also includes and/or functions as a hub network device or module 130 by physically connecting to an external USB hub or a physical RS232 device via a USB-to-RS232 or Bluetooth®-to-RS232 adapter, or by running one or more software applications and/or software modules in the training device 105 to function as a smart device or a Bluetooth® hub. In some embodiments, the training device 105 and the plurality of wearable devices, for example, 115-1, 115-2, 115-3 . . . 115-n are configured to establish the communication and communicate with each other in one or more frequency bands including, but not limited to, the 2.4 GHz band. In some embodiments, the training device 105 also includes at least one specialized chip, micro-controller, or processor including, but not limited to, an ESP32 chip as the hub network device 110 that implements specialized communication protocols including, but not limited to, ESP-NOW protocol to establish the communication and communicate with the plurality of wearable devices, for example, 115-1, 115-2, 115-3 . . . 115-n. The hub network device 130 included in the training device 105 corresponds to any network device, now known or developed in the future developed, configured for establishing a connection and a communication with or between various devices including, but not limited to, the wearable devices, for example, 115-1, 115-2, 115-3 . . . 115-n. The training device 105 and/or the wearable devices, for example, 115-1, 115-2, 115-3 . . . 115-n, correspond to a “paired training device 105” and/or “paired wearable devices 115-1, 115-2, 115-3 . . . 115-n” respectively upon the establishment of the connection and/or the communication between the training device 105 and the wearable devices, for example, 115-1, 115-2, 115-3 . . . 115-n. In some embodiments, upon the establishment of the connection and/or the communication the paired training device 105 is configured to retrieve or obtain wearable device information related to the other paired devices, for example, the paired wearable devices 115-1, 115-2, 115-3 . . . 115-n. In some embodiments, the training device 105 is configured to establish communication with the wearable devices, for example, 115-1 . . . 115-n when the wearable devices are in proximity to the training device 105 within a predefined distance. Examples of the wearable device information include, but are not limited to, a wearable device identifier, a radio frequency identifier, and a radio-frequency address associated with each wearable device, for example, 115-1. It will be apparent to those with ordinary skill in the art that the training device 105 is configured to establish the communication with the wearable devices, for example, 115-1 . . . 115-n and/or retrieve the wearable device information for instances when the wearable devices are powered on for operation. In some embodiments, for instances when the wearable device is powered off and/or inoperable, the training device 105 is configured to retrieve the wearable device information via one or more offline techniques, methods, and/or technologies including, but not limited to, Quick Response (QR) code. For example, in some embodiments, each wearable device, for example, 115-1, is also configured to include at least one QR code provided in least one location on the wearable device. In such embodiments, the at least one QR code includes QR code information associated with the wearable device 115-1. In such embodiments, the training device 105 is configured to retrieve the QR code information by scanning the QR code via one or more input devices including, but not limited to, a scanner (not shown) or a camera (not shown) provided in the training device 105. Examples of the QR code information include, but are not limited to, the wearable device identifier, the radio-frequency identifier, and the radio-frequency address associated with the wearable device, for example, 115-1. It will be apparent to those with ordinary skilled in the art that the training device 105 is configured to, thereby, retrieve the wearable device information via the QR code and/or the retrieved QR code information even when the wearable devices, for example, 115-1 . . . 115-n are powered off and/or inoperable. It will also be apparent to those with ordinary skilled in the art, in such embodiments, when the wearable devices, for example, 115-1 . . . 115-n are powered on for operation subsequently, the training device 105 is configured to establish connection and/or communication with the wearable devices, for example, 115-1 . . . 115-n automatically based on the retrieved wearable device information via the QR code even if the proximity between the training device 105 and the wearable devices, for example, 115-1 . . . 115-n is greater than the predefined distance. In some embodiments, the training device 105 and the wearable devices, for example, 15-1 . . . 115-n communicate with each other as a transmitter-receiver pair. For example, the training device 105 is configured to operate as the transmitter and transmit information to the wearable devices, for example, 115-1 . . . 115-n based on the retrieved wearable device information, for example, the radio-frequency address, and each of the wearable devices, for example, 115-1 . . . 115-n are configured to operate as the receiver to receive the transmitted information from the training device 105. In some embodiments, the training device 105 is configured to support connection or pairing with a predefined number of devices, for example, the wearable devices, for example, 115-1, 115-2, 115-3 . . . 115-n. The predefined number of devices is herein referred to as a ‘hub network capacity’ of the training device 105. In accordance with various embodiments, the training device 105 includes one or more communication ports to receive and transmit the network communication signals from various devices including, but not limited to, the wearable devices, for example, 115-1 . . . 115-n.

In some embodiments, the training device 105 is also configured to established the connection and/or communication with the wearable devices, for example, 115-1, 115-2, 115-3 . . . 115-n via the server 120. In embodiments, the server 120 is configured to store at least one server information associated with the training device 105 and/or the wearable devices, for example, 115-1, 115-2, 115-3 . . . 115-n. Examples of the server information include, but are not limited to, wearable device identification of each wearable device of the wearable devices, for example, 115-1, 115-2, 115-3 . . . 115-n, a player associated with a corresponding wearable device, for example, 115-1, a previous pairing or association between the training device 105 and one or more of the wearable devices, for example, 115-1, 115-2, 115-3 . . . 115-n, one or more training sessions conducted using the training device 105 and the wearable devices, for example, 115-1, 115-2, 115-3 . . . 115-n, data associated with the one or more training sessions conducted, and one or more images and/or video content associated with the training sessions and/or the players captured by the image capturing device(s) 125. In some embodiments, the training device 105 is also configured to established the connection and/or communication with the wearable devices, for example, 115-1, 115-2, 115-3 . . . 115-n via the server 120 based on the server information related to one or more previous pairing or association between the training device 105 and one or more of the wearable devices, for example, 115-1, 115-2, 115-3 . . . 115-n. It will be apparent to those with ordinary skill in the art that the communication between the training device 105 and the wearable devices, for example, 115-1, 115-2, 115-3 . . . 115-n via the server 120 is established when both the training device 105 and the wearable devices, for example, 115-1, 115-2, 115-3 . . . 115-n are in communication with the server 120 via the network 110. In some embodiments, the system 100 also includes one or more additional devices including, but not limited to, routers (not shown) to enable the communication between the server 120 and the training device 105 and/or the wearable devices, for example, 115-1, 115-2, 115-3 . . . 115-n.

In some embodiments, the training device 105 is also configured to establish the connection and/or communication with the wearable devices, for example, 115-1, 115-2, 115-3 . . . 115-n via the server 120 based the at least one QR code provided in the wearable devices, for example, 115-1 . . . 115-n. In such embodiments, the at least one QR code includes information associated with the server 120 that can be retrieved by scanning the QR code via one or more other devices including, but not limited to, the training device 105. In such embodiments, the training device 105 is also configured to provide the retrieved information from the QR code to server 120 via the network 110 and retrieve the information associated with the wearable device, for example, 115-1. In such embodiments, the training device 105 is also configured to establish the connection and/or communication with the wearable device 115-1 based on the retrieved information associated with the wearable device, for example, 115-1 from the server 120. In some embodiments, each wearable device, for example, the wearable device 115-1 of the wearable devices, for example, 115-1, 115-2, 115-3 . . . 115-n, includes a plurality of hardware and software components for performing a plurality of functions respectively. For purposes of clarity and understanding, the components of the wearable device 115-1 are described herein and it will be appreciated by those skilled in the art that other wearable devices, for example, 115-2, 115-3 . . . 115-n may include similar components that perform similar functions.

FIG. 2 depicts the wearable device 115-1 in an exemplary and simplified manner. It will be appreciated that each of the wearable devices 115 comprise the same or similar component structure. The wearable device 115-1 includes, among other components, a wearable device transceiver 205-1, a wearable device processor 210-1, a wearable device power source 215-1, at least one wearable device visual indicator(s) 220-1, a wearable device memory 225-1, and at least one wearable device sensor 230-1, herein referred to as ‘wearable device sensor(s) 230-1’. Although not shown, a person skilled in the art would appreciate that the wearable device 115-1 can also include additional components including, but not limited to, a wearable device display with a wearable device user interface, a wearable device memory 225-1, and/or a wearable device storage unit. In some embodiments, the wearable device 115-1 can also include one or more additional wearable output devices including, but not limited to, wearable device speakers, a wearable device haptic output, or any other output mechanism now known or developed in the future. In addition, in some embodiments, the wearable device 115-1 can further include a serial port, a parallel port, an infrared (IR) interface, a universal serial bus (USB) interface and/or any other interface herein known or developed in the future.

The components of the wearable device 115-1, including, but not limited to, the wearable device transceiver 205-1, the wearable device processor 210-1, wearable device power source 215-1, wearable device visual indicator(s) 220-1, the wearable device memory 225-1, the wearable device sensor(s) 230-1, the wearable device display, and the wearable output devices cooperate with one another to enable operations of the wearable device 115-1. Each component can communicate with one another via a local interface (not shown). The local interface can include, but is not limited to, one or more buses or other wired or wireless connections, as is known in the art. The local interface can have additional elements, which are omitted for simplicity, such as controllers, buffers (caches), drivers, repeaters, and receivers, among many others, to enable communications. Further, the local interface can include address, control, and/or data connections to enable appropriate communications among the aforementioned components.

As illustrated, the wearable device 115-1 in the exemplary embodiment includes the wearable device transceiver 205-1 to provide the one or more inputs to and receive the one or more outputs from other devices, such as, the training device 105 via the network 110 and/or the server 120. The wearable device transceiver 205-1 includes a transmitter circuitry and a receiver circuitry to enable the wearable device 115-1 to communicate data to and acquire data from the training device 105. In this regard, the transmitter circuitry includes appropriate circuitry to provide the one or more inputs, such as, but not limited to, feedback, acknowledgements, wearable device identification, player name, and player identification to the training device 105. Similarly, the receiver circuitry includes appropriate circuitry to receive the one or more outputs, such as, but not limited to, input signals to illuminate the wearable device visual indicator(s) 220-1 from the training device 105. It will be appreciated by those of ordinary skill in the art that the wearable device 115-1 can include a single wearable device transceiver 205-1 as shown, or alternatively separate transmitting and receiving components, for example but not limited to, a transmitter, a transmitting antenna, a receiver, and a receiving antenna. In some embodiments, the wearable device 115-1 can be configured to receive input from the training device 105 via the wearable device transceiver 205-1, and/or provide system output via one or more output devices including, but not limited to, the wearable device visual indicator(s) 220-1, the wearable device display, the wearable device speakers, or the wearable device haptic output, or any other output mechanism now known or developed in the future.

The wearable device memory 225-1 is a non-transitory memory configured to store a set of instructions that are executable by the wearable device processor 210-1 to perform predetermined operations. For example, the wearable device memory 225-1 can include any of the volatile memory elements (for example, random access memory (RAM), nonvolatile memory elements (for example, read only memory (ROM), and combinations thereof. Moreover, the wearable device memory 225-1 can incorporate electronic, magnetic, optical, and/or other types of storage media. In some embodiments, the wearable device memory 225-1 is also configured to store files, such as but not limited to, the wearable device identification, player details including, but not limited to, the player name and the player identification.

The wearable device sensor(s) 230-1 includes, but not limited to, motion sensors, physiological sensors, environmental sensors, and other biochemical sensors. Examples of the motion sensors include, but not limited to, accelerometers, gyroscopes, and Inertial Measurement Units (IMUs). Examples of the physiological sensors include, but not limited to, heart rate monitors, SpO2 sensors, and electrocardiogram (ECG)/electroencephalogram (EEG) sensors. Examples of the environmental sensors include, but not limited to, temperature, humidity, and barometric pressure sensors. In some embodiments, the wearable device sensor(s) 230-1 is configured to capture one or more parameters including, but not limited to, heart rate, pulse-rate, blood oxygen saturation, speed of movement(s), and body temperature of the player using the wearable device 115-1. In some embodiments, the wearable device sensor(s) 230-1 is configured to capture the one or more parameters associated with the player using the wearable device 115-1 during one or more activities being performed by the player.

The wearable device processor 210-1 can include one or more microprocessors, microcontrollers, DSPs (digital signal processors), state machines, logic circuitry, or any other device or devices that process information or signals based on operational or programming instructions. The wearable device processor 210-1 can be implemented using one or more controller technologies, such as Application Specific Integrated Circuit (ASIC), Reduced Instruction Set Computing (RISC) technology, Complex Instruction Set Computing (CISC) technology, or any other similar technology now known or in the future developed. The wearable device processor 210-1 is configured to cooperate with other components of the wearable device 115-1 to perform operations pursuant to communications and the one or more instructions received from the training device 105. The wearable device processor 210-1 is configured to execute the instructions stored in the wearable device memory 225-1 to perform the predetermined operations, for example, illuminating the wearable device visual indicator(s) 220 in one or more colors in response to the inputs received from the training device 105, via the wearable device transceiver 205-1, to display the colors. In some embodiments, the wearable device power source 215-1 is configured to be switched on or activated, via wearable device processor 210-1, to provide electrical power to the wearable device visual indicator(s) 220-1 in order to illuminate the wearable device visual indicator(s) 220-1. It will be appreciated by those with ordinary skill in the art that the wearable device power source 215-1 can correspond to a plurality of wearable device batteries or battery packs coupled with each other. In some embodiments, the plurality of wearable device batteries or battery packs in the wearable device 115-1 are swappable based on a battery status of each wearable device battery in the plurality of wearable device batteries or battery packs. In accordance with various embodiments, the wearable device 115-1 is also configured to constantly monitor information including, but not limited to, a state of charge and/or a count of charge and discharge cycles of the battery and/or the battery pack and indicate the monitored information via a wearable device display (not shown) and/or via the one or more visual indicators provided in the wearable device 115-1. In some embodiments, wearable device 115-1 is also configured to provide at least one recommendation to charge and/or change the battery and/or the battery pack based on the monitoring via the wearable device display (not shown) and/or via the one or more visual indicators provided in the wearable device 115-1. For example, the wearable device 115-1 is also configured to illuminate at least one visual indicator provided in the wearable device 115-1 in red to indicate that the wearable device 115-1 is to be charged. In some embodiments, the wearable device is also configured to receive the monitored information and/or the at least one recommendation via the wearable device transceiver 205-1 and the training device 105. In some embodiments, the components of the wearable device 115-1 can be separated from each other and positioned at different locations in the wearable device 115-1. For example, the wearable device 115-1 can correspond to a smart jersey including the wearable device power source 215-1 and the wearable device visual indicator(s) 220-1 separate from each other and provided at different locations in the smart jersey. In some embodiments, the wearable device processor 210-1 is also configured to receive one or more inputs from the wearable device sensor(s) 230-1 and provide the received input(s) to the training device 105 via the wearable device transceiver 205-1 and the network 110. In some embodiments, the wearable device 115-1 also includes the at least one Quick-Response (QR) code provided at one or more locations including, but not limited to, an exterior surface of the wearable device 115-1. In such embodiments, the at least one QR code includes the QR code information associated with the wearable device 115-1 that can be retrieved by scanning the QR code via one or more other devices including, but not limited to, the training device 105 to establish communication with the wearable device 115-1 directly or via the server 120.

In some embodiments, the training device 105 (see FIG. 1) interfaces directly with the wearable device 115-1 and/or the plurality of other wearable devices, for example, 115-2, 115-3 . . . 115-n via the network 110. In some embodiments, the training device 105 is also configured to establish communication with the wearable device 115-1 and/or the plurality of other wearable devices, for example, 115-2, 115-3 . . . 115-n via the QR code and/or or via the server 120. In an exemplary embodiment, the training device 105 operates as an interface for the instructor/coach to interact with the players associated with the wearable devices, for example, 115-1, 115-2, 115-3 . . . 115-n. The coach utilizes the training device 105 to provide one or more inputs, such as but not limited to, one or more training device communication signals to the wearable devices, for example, 115-1, 115-2, 115-3 . . . 115-n, directly to illuminate the wearable device visual indicator(s), for example, 220-1, 220-2, 220-3 . . . 220-n of each of the plurality of wearable devices, for example, 115-1, 115-2, 115-3 . . . 115-n, to provide one or more visual cues to the players via the wearable device visual indicator(s), for example, 220-1, 220-2, 220-3 . . . 220-n respectively. Examples of the visual cues include, but are not limited to, colors, text, patterns, images, and video. The training device 105 includes a plurality of electrical and electronic components, providing power, operational control, communication, and the like within the training device 105. The various components of the training device 105 will now be described hereinafter.

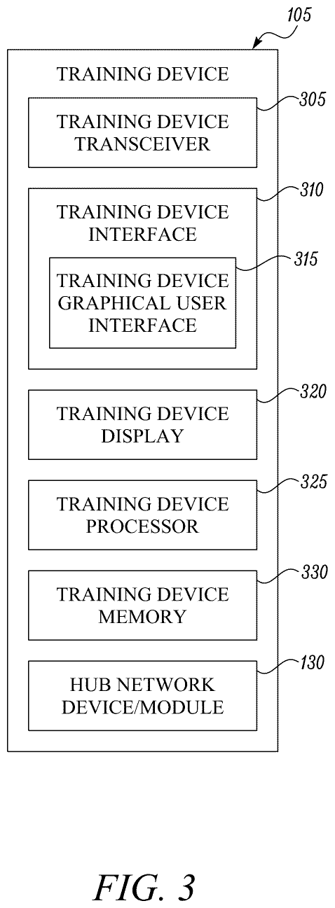

FIG. 3 depicts the training device 105 in an exemplary and simplified manner. However, it should be appreciated by those of ordinary skill in the art that a practical embodiment can include additional components and suitably configured logic to support known or conventional operating features that are not described in detail herein. It will further be appreciated by those of ordinary skill in the art that the training device 105 alternatively can function within a remote server, cloud computing device, or any other local or remote computing mechanism now known or developed in the future. Although the training device 105 is shown and described to be implemented within a single computing device, it can be contemplated that the one or more components of the training device 105 can alternatively be implemented in a distributed computing environment.

The training device 105 includes, among other components, the hub network device or module 130, a training device transceiver 305, a training device interface 310 including a training device graphical interface 315, a training device display 320, a training device processor 325, and a training device memory 330. The components of the training device 105, including the hub network device or module 130, the training device transceiver 305, the training device interface 310, the training device display 320, the training device processor 325, and the training device memory 330, cooperate with one another to enable operations of the training device 105. Each component can communicate with one another via a local interface (not shown). The local interface can be, such as, but not limited to, one or more buses or other wired or wireless connections, as is known in the art. The local interface can have additional elements, which are omitted for simplicity, such as controllers, buffers (caches), drivers, repeaters, and receivers, among many others, to enable communications. Further, the local interface can include address, control, and/or data connections to enable appropriate communications among the aforementioned components.

The training device 105, in the exemplary embodiment, includes the training device transceiver 305 to provide the one or more inputs to and receive the one or more outputs from the wearable devices, for example, 115-1, 115-2, 115-3 . . . 115-n, directly, via the network 110 or via the server 120. The training device transceiver 305 includes a transmitter circuitry and a receiver circuitry to enable the training device 105 to communicate data or instructions to and acquire data from wearable devices, for example, 115-1, 115-2, 115-3 . . . 115-n, directly, via the network 110 or via the server 120. In this regard, the transmitter circuitry includes appropriate circuitry to provide the one or more inputs, such as, but not limited to, one or more signals to the wearable devices, for example, 115-1, 115-2, 115-3 . . . 115-n, to illuminate the wearable device visual indicator(s), for example, 220-1 of the wearable devices, for example, 115-1, 115-2, 115-3 . . . 115-n, and provide the visual cues respectively. Similarly, the receiver circuitry includes appropriate circuitry to receive the one or more outputs including but not limited to, wearable device identification, player name, player identification, and any other information related to the players and/or the wearable devices, for example, 115-1, 115-2, 115-3 . . . 115-n from the wearable devices, for example, 115-1, 115-2, 115-3 . . . 115-n. It will be appreciated by those of ordinary skill in the art that the training device 105 can include a single training device transceiver 305 as shown, or alternatively separate transmitting and receiving components, for example but not limited to, a transmitter, a transmitting antenna, a receiver, and a receiving antenna.

The training device interface 310 is configured to receive one or more instructor/coach inputs and/or to provide one or more system outputs to the instructor/coach and/or to the wearable devices, for example, 115-1, 115-2, 115-3 . . . 115-n, via the network 110 or via the server 120. The instructor/coach inputs can be provided via a keyboard, a touch screen display (such as, the training device display 320), a camera, a touch pad, a microphone, a recorder, a mouse or any other user input mechanism now known or developed in the future. It will be appreciated by those of ordinary skill in the art that the training device 105 can also be configured to provide the instructor/coach inputs to the server 120 such that the instructor/coach inputs and any other input information including, but not limited to, input fields, selection options, and questions related to or associated with the instructor/coach inputs, is stored by the server 120. In such embodiments, the training device 105 can be configured to retrieve, via the training device processor 325, the instructor/coach inputs and the input information related to or associated with the instructor/coach inputs from the server 120 and present the retrieved instructor/coach inputs and/or the input information via one or more training device output devices including, but not limited to, a display device, such as the training device display 320, training device speakers, training device haptic output components, or any other output means or mechanism now known or developed in the future. The training device interface 310 can further include a serial port, a parallel port, an infrared (IR) interface, a universal serial bus (USB) interface and/or any other interface herein known or developed in the future.

In some embodiments, the training device graphical user interface 315 of the training device interface 310 is configured to receive and display communication received from the wearable devices, for example, 115-1, 115-2, 115-3 . . . 115-n, via the network 110 or via the server 120. In some embodiments, the training device graphical user interface 315 is also configured to receive the instructor/coach input and provide instructions to the wearable devices, for example, 115-1, 115-2, 115-3 . . . 115-n, via the network 110 or via the server 120. The training device graphical user interface 315 can be an application or web portal or any other suitable interface now known or developed in the future for conducting a training session for the players. The training device graphical user interface 315 includes one or more of graphical elements associated with the training session. The graphical elements can include, but not limited to one or more of graphical icons, control buttons, timelines, selection boxes, progress indicators, pull-down menus, on-off checkmarks, scroll bars, windows, window edges, toggle buttons, and/or forms. The graphical elements can be used in conjunction with text to prompt the instructor/coach for an input, respond to instructor/coach actions, or display information to the instructor/coach in response to the one or more inputs, feedback or instructions received from the wearable devices, for example, 115-1, 115-2, 115-3 . . . 115-n.

The training device display 320 is configured to display data, images, and the like. The training device display 320 includes a display screen or a computer monitor or any other display mechanism now known or in the future developed. In accordance with some embodiments, the training device display 320 is configured to display the training device graphical user interface 315 associated with the conducting of one or more training sessions for the players.

The training device memory 330 is a non-transitory memory configured to store a set of instructions that are executable by the training device processor 325 to perform predetermined operations. For example, the training device memory 330 can include any of the volatile memory elements (for example, random access memory (RAM), nonvolatile memory elements (for example, read only memory (ROM), and combinations thereof. Moreover, the training device memory 330 can incorporate electronic, magnetic, optical, and/or other types of storage media. In some embodiments, the training device memory 330 is also configured to store files, such as but not limited to, wearable device identification, player names, player identification, player historical performance, predefined activities, game modes, predefined activity definitions, rules, strategies, player ratings, scores, and any other information related to the players, the wearable devices, for example, 115-1, 115-2, 115-3 . . . 115-n. In some embodiments, the training device memory 330 is also configured to store one or more machine learning, artificial intelligence, logical, and/or conditional modules, algorithms, and/or models.

The training device processor 325 can include one or more microprocessors, microcontrollers, DSPs (digital signal processors), state machines, logic circuitry, or any other device or devices that process information or signals based on operational or programming instructions. The training device processor 325 can be implemented using one or more controller technologies, such as Application Specific Integrated Circuit (ASIC), Reduced Instruction Set Computing (RISC) technology, Complex Instruction Set Computing (CISC) technology, or any other similar technology now known or in the future developed. The training device processor 325 is configured to cooperate with other components of the training device 105 to perform operations. In some embodiments, the training device processor 325 is also configured to execute and/or implement the one or more machine learning, artificial intelligence, logical, and/or conditional operations, modules, algorithms, and/or models to perform operations. In some embodiments, the training device processor 325 is also configured to access, retrieve, execute, and/or implement the one or more instructions and/or the one or more machine learning, artificial intelligence, logical, and/or conditional operations, modules, algorithms, and/or models stored in the server 120 (see FIG. 1) to perform operations. The training device processor 325 is configured to execute the instructions stored in the training device memory 330 to perform the predetermined operations, for example, the detailed functions of the training device 105 for conducting a training session for the players will be described hereinafter.

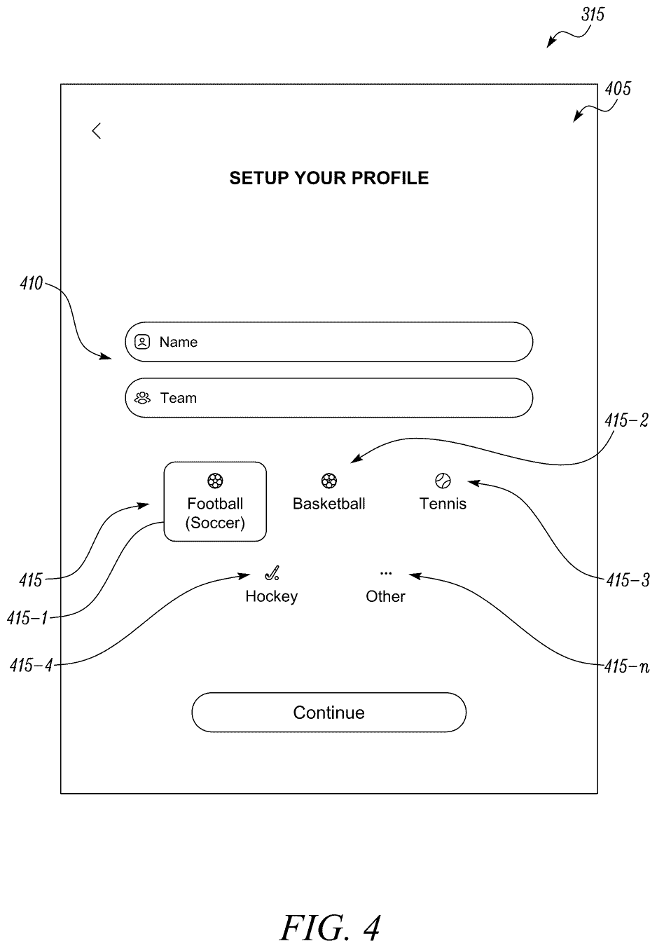

FIGS. 4 through 6 illustrate exemplary graphical user interfaces of the training device of FIG. 3 for establishing communication with the wearable devices, for example, 115-1, 115-2, 115-3 . . . 115-n, of the players respectively.

As illustrated in FIG. 4, the training device 105 is configured to display, via the training device graphical user interface 315, for example, a registration user interface 405 to receive one or more instructor inputs from the instructor/coach managing the training sessions. In an exemplary embodiment, the registration user interface 300 includes one or more registration fields 410 related to instructor/coach details. Examples of the instructor/coach details include, but not limited to, instructor/coach name, team name associated with the instructor/coach and one or more sport icons, for example, 415-1, 415-2, 415-3, 415-4 . . . 415-n, associated with one or more sporting activities including, but not limited to, football, soccer, tennis, basketball, and any other team activity now known or in the future developed. In some embodiments, the training device 105 is configured to receive, via the training device processor 325, the instructor/coach inputs corresponding to the registration fields 410 and the sport icons, for example, 415-1 . . . 415-n from the instructor/coach via the training device graphical user interface 315 or any other input mechanism provided in the training device 105. For example, the coach can select a sport icon 415-1 for football/soccer indicating that the training session corresponds to the sport of football/soccer. In some embodiments, the training device 105 can be configured to store, via the training device processor 325, the instructor/coach inputs received corresponding to the registration fields 410 and the sport icon 415-1 in the training device memory 330. It will be appreciated by those of ordinary skill in the art that the training device 105 can also be configured to provide, via the training device processor 325, the received instructor/coach inputs to the server 120 (see FIG. 1) such that the instructor/coach inputs corresponding to the registration fields 410 and the sport icon 415-1 selected by the coach can be retrieved by the training device 105 from the remote server, via the training device processor 325 and the training device transceiver 305. In some embodiments, the training device 105 can be configured to determine, via, for example, the training device processor 325, the subsequent set of graphical elements or interfaces with different graphic elements respectively to be displayed on the training device graphical user interface 315 based on the received instructor/coach inputs. In such embodiments, the interfaces and/or the graphical elements to be displayed based on the received instructor/coach input corresponding to each sport icon of the sport icons 415 can be different with respect to each other. For example, the training device 105 can be configured to determine, via, for example, the training device processor 325, a first interface including a first set of graphical elements to be displayed in response to the received instructor/coach input corresponding to the sport icon 415-1 representing ‘Football(soccer)’ and a second interface including a second set of graphical elements to be displayed in response to the received instructor/coach input corresponding to the sport icon 415-2 representing ‘Basketball’.

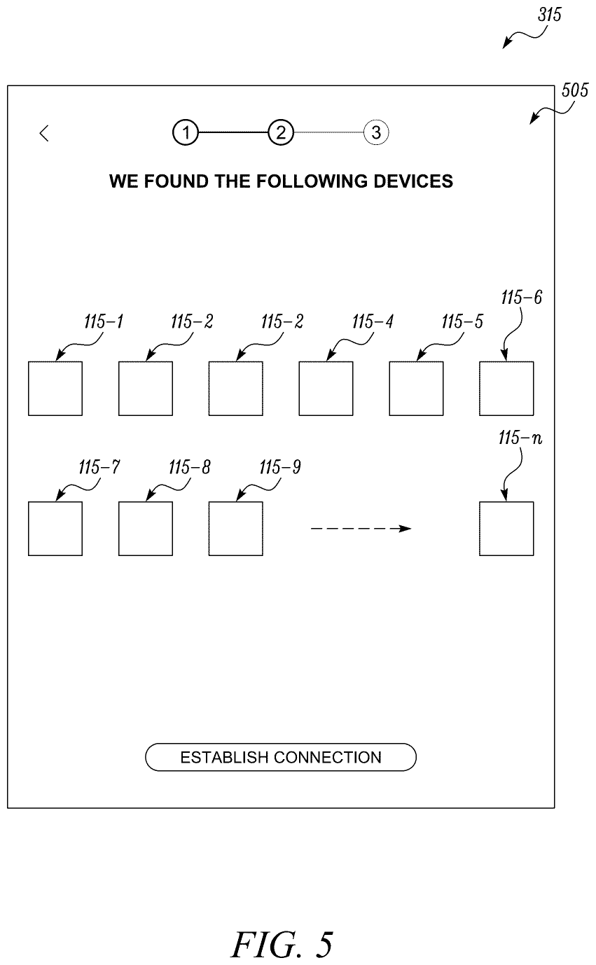

FIG. 5 illustrates the training device graphical user interface 315 of the training device of FIG. 3 for identifying and automatically displaying the wearable devices, for example, 115-1, 115-2, 115-3 . . . 115-n, connected with the training device 105 via the network 110 (see FIG. 1) or via the server 120 (see FIG. 1). In accordance with various embodiments, the training device 105 is configured to detect, via the training device transceiver 305 and the hub network device or module 130 (see FIG. 1), the wearable devices, for example, 115-1, 115-2, 115-3 . . . 115-n and establish a connection/communication, or pair with the wearable devices, for example, 115-1, 115-2, 115-3 . . . 115-n. In some embodiments, the training device 105 is configured to detect and/or identify the wearable devices, for example, 115-1, 115-2, 115-3 . . . 115-n when the wearable devices, for example, 115-1, 115-2, 115-3 . . . 115-n are in proximity, for example, within one square meter of the training device 105. In some embodiments, the training device is also configured to establish the connection/communication or pair with the wearable device, for example, 115-1 . . . 115-n by scanning the QR code provided in the wearable devices and using the QR code information retrieved from the QR code based on the scanning to establish the communication with the wearable devices. In such embodiments, the training device 105 is configured to establish the communication with the wearable devices, for example, 115-1 . . . 115-n directly based on the retrieved QR code information, for example, the radio-frequency address associated with the wearable devices, or via based on the wearable device information, for example, the radio-frequency address retrieved from the server 120 using the QR code information. In some embodiments, the training device 105 is also configured to transmit and receive the network communication signal, for example, but not limited to, a Bluetooth® signal via the training device transceiver 305. In some embodiments, the training device 105 is configured to receive the instructor/coach input to enable the training device transceiver 305 to receive and/or transmit the network communication signal. In accordance with various embodiments, the training device 105 is configured to receive, via the training device transceiver 305 and/or the hub network device or module 130, the network communication signal from the wearable devices, for example, 115-1, 115-2, 115-3 . . . 115-n and detect the wearable devices, for example, 115-1, 115-2, 115-3 . . . 115-n based on the received network communication signal. In some embodiments, the training device 105 is configured to display the detected wearable devices, for example, 115-1, 115-2, 115-3 . . . 115-n via, for example, a network connection user interface 505 provided on the training device graphical user interface 315. In some embodiments, the training device 105 is configured to establish connection and/or communication with the detected wearable devices, for example, 115-1, 115-2, 115-3 . . . 115-n displayed on the training device graphical user interface 315 in response to the instructor/coach input. It will be appreciated by those with ordinary skill in the art that the training device 105 can also be configured to automatically connect to or pair with the wearable devices, for example, 115-1, 115-2, 115-3 . . . 115-n upon detection of the wearable devices, for example, 115-1, 115-2, 115-3 . . . 115-n via the network communication signal.

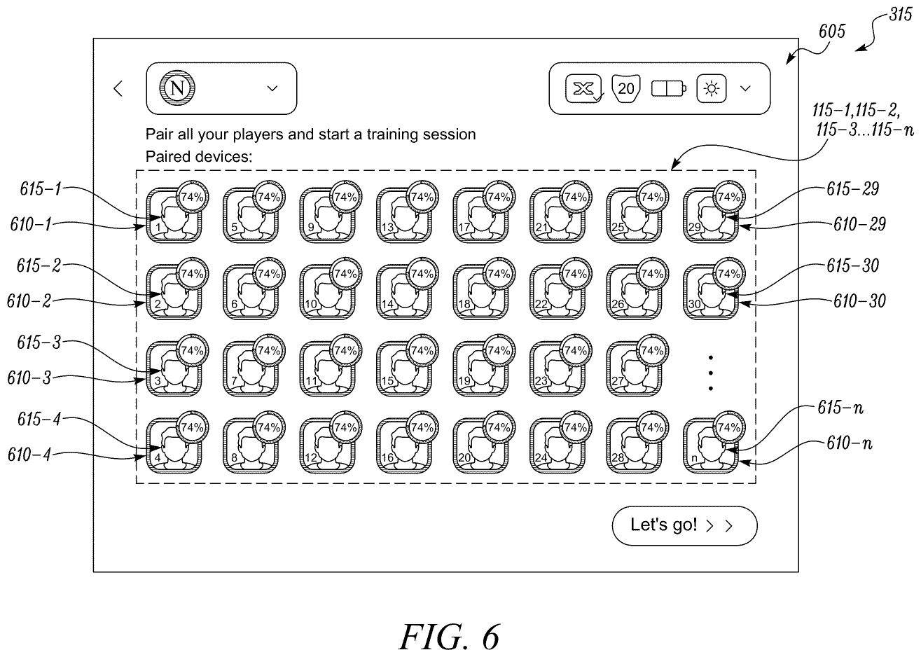

FIG. 6 illustrates the training device graphical user interface 315 of the training device of FIG. 3 for identifying the wearable devices, for example, 115-1, 115-2, 115-3 . . . 115-n, directly connected to the training device 105 via the network 110. In an exemplary embodiment, the training device 105 is configured to display, via the training device graphical user interface 315, a plurality of icons, for example, 610-1, 610-2, 610-3 . . . 610-n correspondingly representing the players, for example, 615-1, 615-2, 615-3 . . . 615-n associated with respective wearable devices, for example, 115-1, 115-2, 115-3 . . . 115-n. In some embodiments, the training device 105 is configured to receive, via the training device transceiver 305, wearable device information related to the wearable devices, for example, 115-1, 115-2, 115-3 . . . 115-n, directly from the wearable devices, for example, 115-1, 115-2, 115-3 . . . 115-n. In some embodiments, the training device 105 is configured to receive, via the training device transceiver 305, wearable device information related to the wearable devices, for example, 115-1, 115-2, 115-3 . . . 115-n upon the establishment of the connection/communication with the wearable devices, for example, 115-1, 115-2, 115-3 . . . 115-n. The wearable device information can include, but is not limited to, wearable device name, wearable device identification, wearable device battery status, player name, and player identification. In some embodiments, the training device 105 is configured to display the wearable devices, for example, 115-1, 115-2, 115-3 . . . 115-n, directly connected with the training device 105, via, for example, a paired players user interface 605 provided on the training device graphical user interface 315. In some embodiments, the training device 105 is also configured to display, via the training device graphical user interface 315, the received wearable device information corresponding the wearable devices, for example, 115-1, 115-2, 115-3 . . . 115-n, respectively. In some embodiments, the training device 105 is also configured to assign an identification of the at least one player with a corresponding wearable device, for example, 115-1 of the wearable devices, for example, 115-1, 115-2, 115-3 . . . 115-n, upon the establishment of the communication with the corresponding wearable device, for example, 115-1 of the at least one player. In some embodiments, the training device 105 is also configured to determine the identification of at least one player associated with the corresponding wearable device, for example, 115-1 based on at least one previous assignment of the at least one player with the corresponding wearable device, for example, 115-1 and/or a previous established connection/communication with the corresponding wearable device, for example, 115-1 of the at least one player. In accordance with various embodiments, the training device 105 is also configured to constantly monitor information including, but not limited to, a state of charge and/or a count of charge and discharge cycles of the battery and/or the battery pack provided in each wearable device, for example, 115-1 connected or paired with the training device 105. In some embodiments, training device 105 is also configured to provide a recommendation to charge and/or change the battery and/or the battery pack provided in each wearable device, for example, 115-1 based on the monitoring via the training device graphical user interface 315. In some embodiments, training device 105 is also configured to provide an alert signal to the one or more wearable devices, for example, 115-1 . . . 115-n via the training device transceiver 305 to indicate the monitored state of charge and/or to indicate the recommendation to charge and/or change the battery and/or the battery pack.

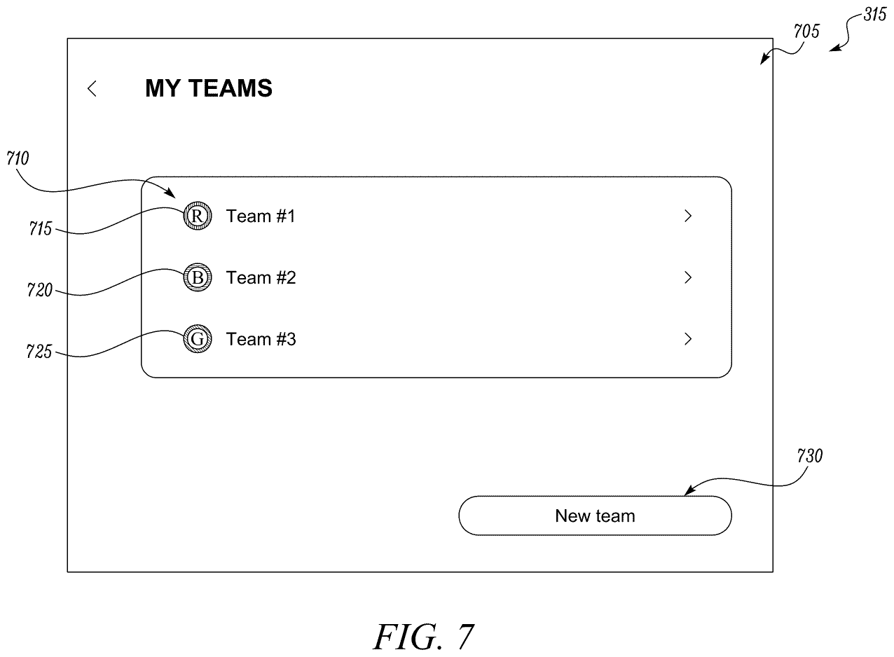

FIGS. 7 through 12 illustrate graphical user interfaces of the exemplary training device of FIG. 3 for conducting one or more training sessions for the players, for example, 615-1 . . . 615-n (see FIG. 6).

As illustrated in FIG. 7, in some embodiments, the training device 105 is configured to create, via the training device processor 325, a set of randomized virtual teams 710 of the players, for example, 615-1 . . . 615-n (see FIG. 6) associated with the wearable devices 115-1, 115-2, 115-3 . . . 115-n (see FIG. 6). In an exemplary embodiment, the training device 105 is configured to display, via, for example, a team creation user interface 705 provided on the training device graphical interface 315, to enable the instructor/coach to create the set of randomized virtual teams 710 including, but not limited to, a first virtual team 715, a second virtual team 720, and a third virtual team 725, via, for example, a graphical team creation button 730. In some embodiments, the training device 105 is configured to receive one or more instructor/coach inputs related to the teams to be created, via the training device graphical interface 315 or any other input mechanism. Examples of the instructor/coach inputs related to each virtual team include, but are not limited to, team name and a team logo/icon. In accordance with various embodiments, the training device 105 is configured to automatically create, via the training device processor 325, the set of randomized virtual teams based on a count of the connected wearable devices, for example, 115-1, 115-2, 115-3 . . . 115-n, of the players connected and/or identified by the training device 105. In some embodiments, the training device 105 is also configured to automatically create the set of randomized virtual teams based on at least one selection criteria. Examples of the at least one selection criteria include, but are not limited to, at least one historical performance of the plurality of players, at least one historical virtual team of the plurality of players, at least one skill associated with the plurality of players, at least one predefined team composition criteria, at least one predetermined strength, weakness, or a combination thereof of each player of the plurality of players, a randomized selection of the plurality of players, or any combination thereof. In some embodiments, at least one predefined team composition criteria defines the at least one skill, the at least one historical performance, at least one complimentary skill of the at least one skill between the plurality of players, or any combination thereof corresponding to the set of randomized teams to be created.

In some embodiments, a count of the players associated with respective wearable devices 115-1, 115-2, 115-3 . . . 115-n, in each created team of the randomized set of created virtual teams can be different from each other. In some embodiments, the players associated with the respective wearable devices 115-1, 115-2, 115-3 . . . 115-n, included in each created team of the set of randomized created virtual teams is different from each other. In some embodiments, at least one player of the players associated with the respective wearable devices 115-1, 115-2, 115-3 . . . 115-n, is included in a plurality of created virtual teams of the set of randomized created virtual teams. In an exemplary embodiment, the training device 105 is configured to create the set of randomized virtual teams 710 by selecting one or more icons of the plurality of icons, for example, 610-1 . . . 610-n (see FIG. 6) corresponding to the players, for example, 615-1 . . . 615-n (see FIG. 6) in each virtual team. In some embodiments, the training device 105 is configured to apply, via the training device processor 325, at least one artificial intelligence model to create the set of randomized virtual teams 710 including, for example, but not limited to, the first virtual team 715, the second virtual team 720, the third virtual team 725 and/or the set of randomized teams based on historical data including, but not limited to, at least one of previously created virtual teams previously conducted training session with the created virtual teams, and/or player performance.

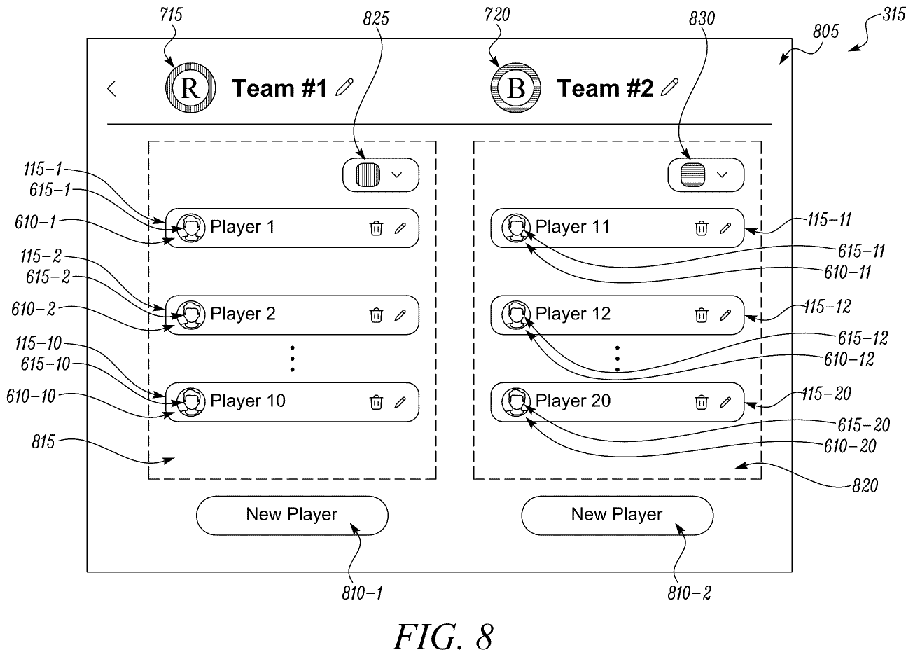

FIG. 8 illustrates an exemplary training device graphical user interface 315 of the training device of FIG. 3 for including one or more players of the players, for example, 615-1 . . . 615-n (see FIG. 6) in each virtual team of the created set of randomized virtual teams 710 (see FIG. 7). In an exemplary embodiment, the training device 105 is configured to display, via, for example, a player selection user interface 805 provided on the training device graphical interface 315, to enable the selection, via, for example, one or more graphical player selection buttons, for example, 810-1, 810-2 and inclusion of one or more subsets of players of the players, for example, 615-1 . . . 615-n associated with respective subsets of wearable devices of the wearable devices, for example, 115-1 . . . 115-n in each virtual team. In some embodiments, a selection of the graphical player selection button 810-1, via the training device graphical user interface 315, causes the training device 105 to display, via the training device processor 325, the paired players user interface 605 (see FIG. 6) and receive the selection of the players, for example, 615-1 . . . 615-n via the paired players user interface 605. In some embodiments, upon receipt of the selection from the paired players user interface 605, the training device 105 is configured to display the player selection user interface 805 including one or more icons of the icons, for example, 610-1 . . . 610-n (see FIG. 6) representing the one or more selected players, via the training device graphical user interface 315.

As an example, the training device 105 is configured to create, via the training device processor 325, the first virtual team 715 (see FIG. 7), based on the selection of a first subset of icons, for example, 610-1, 610-2 . . . 610-10 of the plurality of icons 610-1 . . . 610-n (see FIG. 6) corresponding to a first subset of players, for example, 615-1, 615-2 . . . 615-10 of the plurality of players, for example, 615-1 . . . 615-n (see FIG. 6). The training device 105 is also configured to create, via the training device processor 325, the second virtual team 720 (see FIG. 7), based on a selection of a second subset of icons, for example, 610-11 . . . 610-20, of the plurality of icons 610-1 . . . 610-n corresponding to a second subset of players, for example, 615-11, 615-12 . . . 615-20 of the plurality of players, for example, 615-1 . . . 615-n. In some embodiments, the first subset of selected players, for example, 615-1 . . . 615-10 is different from the second subset of selected players, for example, 615-11 . . . 615-20. In some embodiments, the training device 105 is configured to create the first virtual team 715 and the second virtual team 720 by selectively moving one or more icons of the icons, for example, 610-1 . . . 610-n to a first designated area 815 and a second designated area 820. For example, the training device 105 is configured to create the first virtual team and the second virtual team by moving the corresponding first subset of icons, for example, 610-1, 610-2 . . . 610-10 and the second subset of icons 610-11, 610-12 . . . 610-20 to the first designated area 815 and the second designated area 820 respectively on the training device graphical user interface 315 based on one or more instructor/coach inputs received via the training device graphical user interface 315 or any other input means/mechanism. In some embodiments, the training device 105 is configured to apply, via the training device processor 325, at least one artificial intelligence model to automatically create the first virtual team 715 and the second virtual team 720, based on historical data including, but not limited to, at least one of previously created virtual teams previously conducted training session with the created virtual teams, and/or player performance. In some embodiments, the training device 105 is also configured to edit, via the training device processor 325 and the training device graphical user interface 315, player details including, but not limited to, the player name, and player image/avatar/icon associated with the players upon the selection of the icons, for example, 610-1 . . . 610-n representing the players, for example, 615-1 . . . 615-n in each virtual team. In some embodiments, the training device 105 is also configured to delete, via the training device processor 325 and the training device graphical user interface 315, one or more players selected and included in each virtual team.

In some embodiments, the training device 105 is configured to generate, via the at least one artificial intelligence model, a plurality of predefined activities to be performed by the created set of randomized virtual teams at a plurality of time intervals respectively. In some embodiments, the training device 105 is configured to generate, via the at least one artificial intelligence model, a randomized sequence of the plurality of predefined activities to be performed in a sequence for corresponding time intervals prior to the commencement of a first predefined activity of the plurality of predefined activities. For example, the training device 105 is configured to generate the first predefined activity, the second predefined activity and the third predefined activity to be performed by the players, for example, the first subset of players and the second subset of players, for respective time intervals prior to the commencement of the first predefined activity. In some embodiments, the training device 105 is configured to generate, via the at least one artificial intelligence model, the first predefined activity of the plurality of predefined activities and dynamically generate, via the at least one artificial intelligence model, each subsequent predefined activity of the plurality of predefined activities to be performed after the first predefined activity in real-time. For example, each subsequent predefined activity is dynamically generated prior to, during, or after the commencement of a current predefined activity of the plurality of predefined activities. As an exemplary example, the training device 105 is configured to generate, via the at least one artificial intelligence model, dynamically generate a second predefined activity to be performed by the players, for example, the first and second subset of players prior to, during, or after the commencement of the first predefined activity. Similarly, the training device 105 is configured to generate, via the at least one artificial intelligence model, dynamically generate a third predefined activity to be performed by the players, for example, the first and second subset of players prior to, during, or after the commencement of the second predefined activity.

In some embodiments, the training device 105 is configured to assign, via the at least one artificial intelligence model, the plurality of time intervals corresponding to the plurality of predefined activities prior to the commencement of the first predefined activity. For example, the training device 105 is configured to assign the respective time intervals for performing the first predefined activity, the second predefined activity and the third predefined activity by the players, for example, the first subset of players and the second subset of players, prior to the commencement of the first predefined activity. In some embodiments, the plurality of assigned time intervals is same or different from each other. For example, the assigned first time interval, the assigned second time interval, and the assigned third time interval for performing the first predefined activity, the second predefined activity, and the third predefined activity by the players is same or different. In some embodiments, the training device 105 is also configured to dynamically assign, via the at least one artificial intelligence model, a time interval of the plurality of time intervals corresponding to each predefined activity of the plurality of predefined activities prior to the commencement of each predefined activity in real-time. As an exemplary example, the training device 105 is configured to assign, via the at least one artificial intelligence model, a first interval for performing the first predefined activity by the players, and dynamically assign, via the artificial intelligence model, a second time interval for performing the second predefined activity by the players prior to, during, or after the commencement of the first predefined activity. Similarly, the training device 105 is configured to dynamically assign, via the at least one artificial intelligence model, a third time interval for performing the third predefined activity prior to, during, or after the commencement of the second predefined activity. In some embodiments, the dynamically assigned time interval corresponding to each predefined activity by the players is same or different from each other. For example, the dynamically assigned first time interval, the dynamically assigned second time interval, and the dynamically assigned third time interval for performing the first predefined activity, the second predefined activity, and the third predefined activity by the players are same or different. In some embodiments, the training device 105 is also configured to dynamically modify, via the at least one artificial intelligence model, the plurality of assigned time intervals or the dynamically assigned time intervals during a current predefined activity being performed by the players. For example, the training device 105 is configured to dynamically modify the assigned first time interval during the first predefined activity being performed by the players. Similarly, the training device 105 is configured to dynamically modify the dynamically assigned second time interval corresponding to the second predefined activity during the second predefined activity being performed by the players.

In some embodiments, the training device 105 is configured to automatically commence, via the at least one artificial intelligence model, a first predefined activity of the plurality of predefined activities for the first time interval of the plurality of time intervals. In some embodiments, the training device 105 is configured to associate, via the training device processor 325, the created set of randomized virtual teams 710, for example, the first virtual team 715 and the second virtual team 720, with a plurality of distinct visual cues including, but not limited to, colors, text, patterns, images, and video, respectively to commence one or more of the plurality of predefined activities. For purposes of clarity and understanding, the plurality of visual cues corresponding to a plurality of distinct colors is disclosed herein after. As an example, the training device 105 can be configured to associate, via the training device processor 325, the first virtual team 715 and the first subset of icons, for example, 610-1 . . . 610-10 with a first color 825 based on a selection of the first color 825 among the plurality of colors via the training device graphical user interface 315. Similarly, the training device 105 can also be configured to associate the second virtual team 720 and the second subset of icons, for example, 610-11 . . . 610-20 with a second color 830 based on a selection of the second color 830, via the training device graphical user interface 315. In some embodiments, the training device 105 is configured to apply, via the training device processor 325, at least one artificial intelligence model to automatically associate the created set of randomized virtual teams 710 with the plurality of distinct colors. For example, the training device 105 can be configured to apply, via the training device processor 325, at least one artificial intelligence model to automatically associate the first virtual team 715 and the second virtual team 720 with the first color 825 and the second color 830 respectively. In some embodiments, the training device 105 is further configured to selectively associate, via the training device processor 325, one or more selected players of the players, for example, 615-1 . . . 615-n with plurality of distinct colors respectively, via the artificial intelligence model or via the instructor/coach inputs received corresponding to the icons, for example, 610-1 . . . 610-n representing the players 615, for example, 615-1 . . . 615-n, via the training device graphical user interface 315 or other any input means/mechanism.

In some embodiments, the training device 105 is configured to selectively transmit, via the training device processor 325, the training device transceiver 305, a training device communication signal to the wearable devices, for example, 115-1, 115-2, 115-3 . . . 115-n, associated with the players, for example, 615-1 . . . 615-n to illuminate the wearable device visual indicator(s), for example, 220-1 . . . 220-n (see FIG. 1) of each wearable device in the respective associated color. In some embodiments, the training device 105 is configured to transmit the training device communication signal to the wearable devices, for example, 115-1, 115-2, 115-3 . . . 115-n respectively directly via the network 110 or via the server 120. In some embodiments, the training device 105 may be configured to transmit the training device communication signal to the wearable devices, for example, 115-1, 115-2, 115-3 . . . 115-n to illuminate the wearable device visual indicator(s), for example, 220-1 . . . 220-n (see FIG. 1) of each wearable device for a predefined time interval automatically or based on one or more instructor/coach inputs received via the training device graphical user interface 315 or any other input means/mechanism. In some embodiments, the training device 105 is configured to selectively transmit different training device communication signals to different subsets of paired wearable devices based on the selection of the players, for example, 615-1 . . . 615-n in the created set of randomized virtual teams 710 and the color associated with the each team of the created set of randomized virtual teams 710 respectively. For example, the training device 105 is configured to selectively transmit a first training device communication signal to the first subset of paired wearable devices, for example, 115-1 . . . 115-10 of the wearable devices, for example, 115-1, 115-2, 115-3 . . . 115-n corresponding to the first subset of players, for example, 615-1 . . . 615-10 to illuminate the wearable device visual indicator(s) 220-1 . . . 220-10 of each of the first subset of wearable devices, for example, 115-1 . . . 115-10 in the first color 825 respectively. Similarly, the training device 105 is also configured to transmit, via the training device processor 325, the training device transceiver 305, and the hub network device/module 130, a second training device communication signal to the second subset of wearable devices, for example, 115-11 . . . 115-20 of the wearable devices, for example, 115-1, 115-2, 115-3 . . . 115-n corresponding to the second subset of players, for example, 615-11 . . . 615-20 to illuminate the wearable device visual indicator(s), for example, 220-11 . . . 220-20 of each of the second subset of wearable devices, for example, 115-11 . . . 115-20 with the second color 830 respectively. In some embodiments, the first color can be same as or different from the second color. In some embodiments, the training device 105 is configured to selectively transmit the training device communication signals to the wearable devices 115-1, 115-2, 115-3 . . . 115-n, associated with respective players in each virtual team immediately in response to the association of the color with each virtual team or the icons, for example, 610-1 . . . 610-n (see FIG. 6) representing the players, for example, 615-1 . . . 615-n (see FIG. 6) respectively. In some embodiments, the training device 105 is also configured to selectively transmit, via the training device transceiver 305, a third signal to the at least one wearable device, for example, 115-1 corresponding to at least one player of the players. In some embodiments, the at least one wearable device, for example, 115-1, is configured to illuminate the visual indicator in the at least one color in response to the received third signal. In some embodiments, the training device 105 is configured to define one or more illumination parameters including, but not limited to, a count of the visual indicators to be illuminated in each wearable device, a color intensity of the color, a gradient of the color, a time interval to illuminate each wearable device, and a time interval to change the color to another color, in each training device communication signal. In some embodiments, the training device 105 is configured to receive the illumination parameters and/or one or more instructor/coach inputs corresponding to the illumination parameters via the training device graphical user interface 315 or any other input means/mechanism.

In accordance with various embodiments, the wearable devices 115-1, 115-2, 115-3 . . . 115-n, are configured to receive the training device communication signals from the training device 105 via the network 110 and illuminate, via the respective wearable device processors, for example, 210-1 (see FIG. 2), the wearable device visual indicator(s) 220-1, 220-2, 220-3 . . . 220-n (see FIG. 2) of the wearable devices, for example, 115-1, 115-2, 115-3 . . . 115-n respectively. In some embodiments, the wearable devices, for example, 115-1, 115-2, 115-3 . . . 115-n are configured to illuminate the wearable device visual indicator(s), for example, 220-1 . . . 220-n based on the illumination parameters received from the training device 105, via the network 110. For example, in some embodiments, the wearable devices, for example, 115-1, 115-2, 115-3 . . . 115-n are configured to selectively illuminate, via the respective wearable device processors, for example, 210-1, one or more wearable device visual indicator(s) of each wearable device based on the received training device communication signal. For example, a wearable device 115-1 can include ten light emitting diode (LED) lights and can be configured to selectively illuminate only five light emitting diode (LED) lights based on the received training device communication signal. Similarly, in some embodiments, the wearable devices, for example, 115-1, 115-2, 115-3 . . . 115-n are configured to illuminate the wearable device visual indicator(s), for example, 220-1 . . . 220-n, based on the color intensity and/or gradient defined the received illumination parameters.

In accordance with various embodiments, the illumination of the wearable device visual indicator(s), for example, 220-1 . . . 220-n in the wearable devices, for example, 115-1 . . . 15-n in one or more colors indicates commencement of one or more predefined activities, each representing a training session to be performed by the created set of randomized virtual teams 710 (see FIG. 7) or the player associated with the corresponding wearable device. As an example, in some embodiments, the illumination of the wearable device visual indicator(s), for example, 220-1 . . . 220-10 of the first subset of wearable devices, for example, 115-1 . . . 115-10 and the wearable device visual indicator(s), for example, 220-11 . . . 220-20 of the first subset of wearable devices, for example, 115-11 . . . 115-20 in the first color and the second color respectively indicates commencement of a first predefined activity between the first virtual team 715 or the first subset of players, for example, 615-1 . . . 615-10 and the second virtual team 720 or the second subset of players, for example, 615-11 . . . 615-20. In some embodiments, the first predefined activity corresponds to a game to be played between the first virtual team 715 or the first subset of players, for example, 615-1 . . . 615-10 and the second virtual team 720 or the second subset of players, for example, 615-11 . . . 615-20 for a predefined time interval, for example, the assigned time interval or the dynamically assigned time interval. In some embodiments, the training device 105 is also configured to receive instructor/coach input corresponding the predefined time interval in order to define or change the predefined time interval via the training device graphical user interface 315.

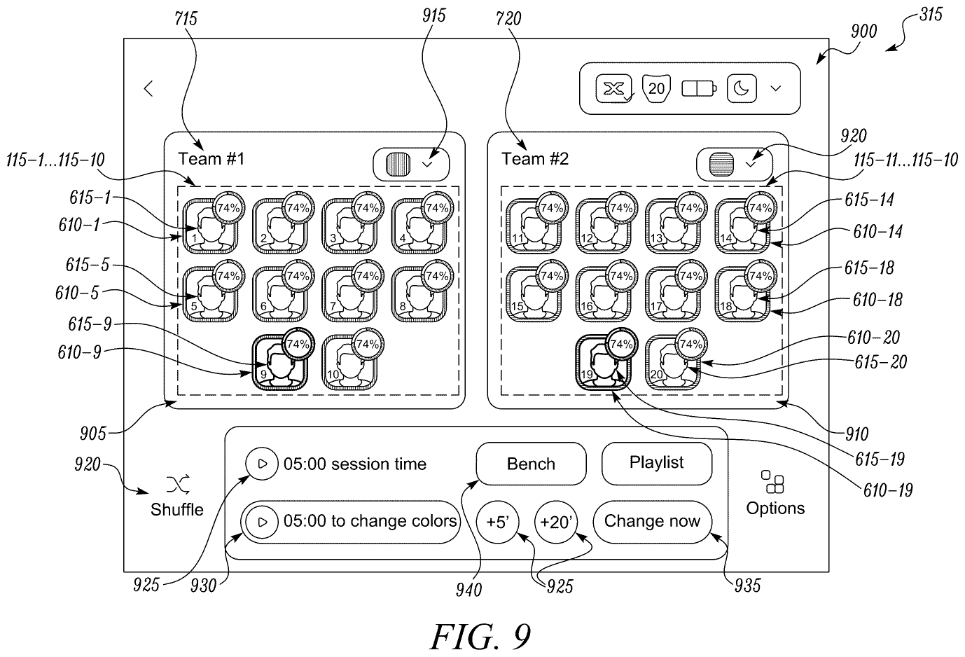

FIG. 9 illustrates an exemplary match session user interface 900 provided on the training device graphical user interface 315 of the training device of FIG. 3 for conducting a first training session for the first virtual team 715 or the first subset of players, for example, 615-1 . . . 615-10, and the second virtual team 720 or the second subset of players, for example, 615-11 . . . 615-20. In some embodiments, the match session corresponds to a sporting game, or any other activity played between at least two virtual teams of the virtual teams, for example, 715, 720, 725 (see FIG. 7). In an exemplary embodiment, the first training session corresponds to a game session or a game mode that includes the first predefined activity. In order to begin the first training session, the training device 105 is configured to associate the first color 825 (see FIG. 8) and the second color 830 (see FIG. 8) with the first virtual team 715 and the second virtual team 720 respectively and transmit the training device communication signals to the first subset of wearable devices, for example, 115-1 . . . 115-10 of first subset of players, for example, 615-1 . . . 615-10, and the second subset of wearable devices, for example, 115-11 . . . 115-20 of the second subset of players, for example, 615-11 . . . 615-20 to illuminate the wearable device visual indicator(s), for example, 220-1 . . . 220-10 of the first subset of wearable devices, for example, 115-1 . . . 115-10 and the wearable device visual indicator(s), for example, 220-11 . . . 220-20 of the second subset of wearable devices, for example, 115-11 . . . 115-20 in the first color the second color respectively to indicate commencement of the first predefined activity. The first predefined activity corresponds to the game to be played between the first virtual team 715 and the second virtual team 720 for the predefined time interval defined and/or displayed, via one or more graphical time interval buttons 925 on the training device graphical user interface 315.

In some embodiments, the training device 105 is also configured to automatically commence, via the at least one artificial intelligence model, at least one subsequent predefined activity of the plurality of predefined activities that is different from the first predefined activity for a subsequent time interval of the plurality of time intervals after or during the first time interval. In some embodiments, the at least one subsequent predefined activity corresponds to a dynamic modification of the first predefined activity including at least one additional rule applied to the first predefined activity, a change in frequency of the first predefined activity, a change in an intensity of the first predefined activity, at least one external stimuli or a change in the at least one external stimuli provided to the first subset of players, the second subset of players, the at least one player, or any combination thereof during or after the first interval of the first predefined activity, or any combination thereof. For example, for instances when the first predefined activity corresponds to one or more exercises to be performed, the at least one subsequent activity corresponds to the change in frequency of the exercises to be performed and/or the change in intensity of each exercise of the exercises to be performed. In some embodiments, the training device 105 is configured to automatically associate, via the training device processor 325, at least one icon, for example, 610-9, 610-19 of the plurality of icons, for example, 610-1 . . . 610-n (see FIG. 6) corresponding to at least one player, for example, 615-9, 615-19 of the plurality of players, for example, 615-1 . . . 615-n (see FIG. 6) with at least one color, for example, a third color, of the plurality of distinct colors to commence the at least one subsequent activity. In some embodiments, the training device 105 is also configured to receive an instructor/coach input, via, for example, one or more graphical color selection dropdown options 915, provided on the training device graphical user interface 315, corresponding to the at least one icon to receive a selection of the third color to be associated with the icon. In some embodiments, the third color is different from the first color 825 (see FIG. 8) and the second color 830 (see FIG. 8). For example, the training device 105 can be configured to associate the icons 610-9, 610-19 representing the players 615-9, 615-19 from the first subset of players, for example, 615-1 . . . 615-10 and the second subset of players, for example, 615-11 . . . 615-20 respectively with the third color that is different from the first color previously associated with the icon 610-9 and the second color previously associated with the icon 610-19. In some embodiments, the third color is same as the first color or the second color and different from the previously associated color of the at least one icon corresponding to the at least one player. In such embodiments, associating the least one icon corresponding to the at least one player with the at least one color, for example, the third color, corresponds to associating the at least one icon with the first subset of icons, for example, 610-1 . . . 610-10 corresponding to the first subset of players, for example, 615-1 . . . 615-10, or the second subset of icons, for example, 610-11 . . . 610-20 corresponding to the second subset of players, for example, 615-11 . . . 615-20. For example, the training device 105 can be configured to associate the icon 610-9 representing the player 615-9 from the first subset of players, for example, 615-1 . . . 615-10 with the third color equivalent to the second color 830 (see FIG. 8) associated with the second subset of icons, for example, 610-11 . . . 610-20 and accordingly, associate the icon 610-9 with the second subset of icons, for example, 610-11 . . . 610-20 corresponding to the second subset of players, for example, 615-11 . . . 615-20. In some embodiments, the training device 105 is configured to associate the icon 610-9 representing the player 615-9 from the first subset of players, for example, 615-1 . . . 615-10 with the at least one color equivalent to the second color automatically by moving the icon 610-9 from a first designated area 905 provided on the training device graphical user interface 315 for the first subset of icons, for example, 610-1 . . . 610-10 to a second designated area 910 provided on the training device graphical user interface 315 for the second subset of icons, for example, 610-11 . . . 610-20. In some embodiments, the training device 105 can receive the instructor/coach input via the training device graphical user interface 315 or any other input means/mechanism, for the movement of the at least one icon from the first designated area 905 to the second designated area 910. In some embodiments, the training device 105 is configured to change the previously associated colors of the icons 610-9, 610-19 with the at least one color, for example, the third color automatically after a predefined time interval, for example, the assigned time interval or the dynamically assigned time interval, defined and/or displayed, via the graphical color change button 930 provided on the training device graphical user interface 315. In some embodiments, the training device 105 is configured to change the previously associated colors of the icons 610-9, 610-19 with the at least one color, for example, the third color in response to an instructor/coach input received via a graphical instructor color change button 935 provided on the training device graphical user interface 315. In some embodiments, the training device 105 is configured to randomly select one or more icons from the first subset of icons, for example, 610-1 . . . 610-10 associated with the first color and associate with the randomly selected icons with the at least one color equivalent to the second color. In some embodiments, the training device 105 is also configured to randomly select one or more icons from the second subset of icons, for example, 610-11 . . . 610-20 associated with the second color and associate with the randomly selected icons with the at least one color equivalent to the first color. In such embodiments, the training device 105 is configured to randomly select the one or more icons via the at least one artificial intelligence model or in response to the instructor/coach input received, via, for example, a graphical shuffle players button 920 provided on the training device graphical user interface 315.

In some embodiments, the training device 105 is configured to associate the at least one icon with the at least one color, for example, the third color such that a count of the first subset of icons, for example, 610-1 . . . 610-10 representing the first subset of players, for example, 615-1 . . . 615-10 is different from a count of the second subset of icons, for example, 610-11 . . . 610-20 representing the second subset of players, for example, 615-11 . . . 615-20 after the association. It will be appreciated by those of ordinary skill in the art that the training device 105 can be configured to associate at least one icon with the at least one color, for example, the third color and/or move the icon from the first designated area 905 to the second designated area 910 after a predefined interval of time or at any given point in time during the first training session. It will also be appreciated by those of ordinary skill in the art that the training device 105 can be configured to identify the at least one icon from the first subset of icons, for example, 610-1 . . . 610-10, the second subset of icons, for example, 610-11 . . . 610-20 or the remaining icons from the plurality of icons, for example, 610-21 . . . 610-n (see FIG. 6).

In some embodiments, the training device 105 is also configured to transmit, via the training device processor 325 and the training device transceiver 305, one or more third training device communication signals to one or more wearable devices, for example, 115-9, 115-19 of the plurality of wearable devices, for example, 115-1, 115-2, 115-3 . . . 115-n associated with the one or more players, for example, 615-9, 615-19 representing the one or more icons, for example 610-9, 610-19 associated with the at least one color, for example, the third color to illuminate the wearable device visual indicator(s), for example, 220-9, 220-19 of respective wearable devices, for example, 115-9, 115-19 associated with the players, for example, 615-9, 615-19 in the third color. In some embodiments, the training device 105 is configured to transmit the third training device communication signals to the wearable devices, for example, 115-9, 115-19 respectively directly via the network 110 or via the server 120. In some embodiments, the training device 105 is configured to apply, via the training device processor 325, at least one artificial intelligence model to automatically identify or randomly select and associate the at least one icon among the plurality of icons, for example, 610-1 . . . 610-n with the at least one color, for example, the third color and transmit the third signal to the wearable device corresponding to the player representing the icon associated with at least one color, for example, the third color. In some embodiments, the artificial intelligence model is trained based on at least one previously conducted training session.

In some embodiments, the illumination of the visual indicators in the at least one color is indicative of the commencement of the at least one subsequent predefined activity by the player representing the icon associated with the at least one color. For example, the illumination of the visual indicators in the third color indicates commencement of a second predefined activity by the player representing the icon associated with the third color. In some embodiments, the second predefined activity corresponds to assigning one or more players, for example, 615-9, 615-19 representing the icons 610-9, 610-19 associated with the third color to rest or withdraw from the first training session. In some embodiments, the training device 105 can also be configured to automatically select, via the training device processor 325, one or more icons, for example, 610-9, 610-19 to rest or withdraw the players 615-9, 615-19 representing the icons 610-9, 610-19 from the first training session after a predefined time interval, for example, the assigned time interval or the dynamically assigned time interval, from the start of the first training session. In some embodiments, the training device 105 can also be configured to selectively rest or withdraw the players, for example, 615-9, 615-19 representing the icons, for example, 610-9, 610-19 from the first training session automatically based on various factors including, but not limited to, at least one input received from the wearable device sensor(s) 230-1 (see FIG. 2) and a performance of one or more players during the training session. In some embodiments, the training device 105 can also be configured to selectively rest or withdraw the players, for example, 615-9, 615-19 representing the icons, for example, 610-9, 610-19 from the first training session based on the selection of the icons 610-9, 610-19 via the training device graphical interface 315 and a selection of a graphical bench player button 940 provided on the training device graphical interface 315. In some embodiments, the illumination of the visual indicator in the third color indicates feedback or a predefined instruction for the at least one player, for example, the player 615-9. For example, the feedback can represent a performance rating of the player. In some embodiments, the second predefined activity corresponds to the at least one player of the players, for example, 615-1 . . . 615-n (see FIG. 6) optionally playing for the first virtual team 715 or the second virtual team 720 as a ‘double agent’ as described in detail hereinafter.

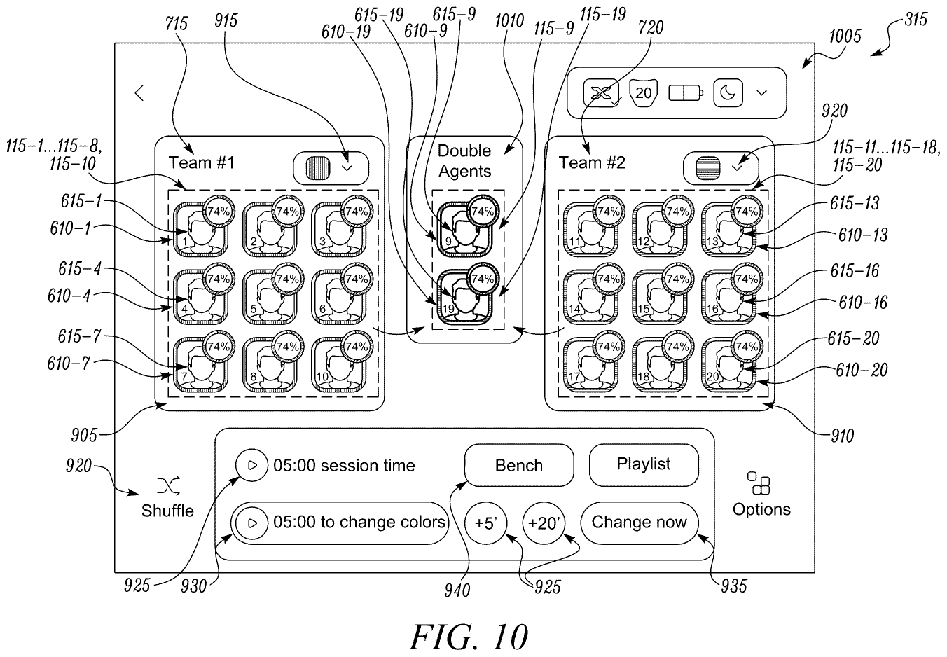

FIG. 10 illustrates an exemplary double agent session user interface 1005 provided on the training device graphical user interface 315 of the training device of FIG. 3 for conducting a second training session or a second predefined activity during the first training session for the first virtual team 715 or the first subset of players, for example, 615-1 . . . 615-n, the second virtual team 720 or the second subset of players, for example, 615-11 . . . 615-20, and the one or more players associated with wearable devices illuminated in the third color. In an exemplary embodiment, the second training session corresponds to a double agent session or a double agent mode that includes the second predefined activity. In order to begin the second training session or the second predefined activity, the training device 105 is configured to associate the third color with the icons, for example, 610-9, 610-19 representing the players 615-9, 615-19 after a predefined time interval, for example, the assigned time interval or the dynamically assigned time interval, from a starting time of the first predefined activity automatically based on the graphical color change button 930 or based on the instructor/coach input received via the graphical instructor color change button 935. The icons 610-9, 610-19 associated with the third color correspond to a third subset of wearable devices, for example, 115-9, 115-19 associated with the players, for example, 615-9, 615-19. In some embodiments, the training device 105 is also configured to display a third designated area 1010 on the training device graphical interface 315 and move the icons, for example, 610-9, 610-19 associated with the third color in the third designated area 1010. In some embodiments, the training device 105 is also configured to associate one or more icons, for example, 610-9, 610-19 with the third color upon movement of the icons from the first designated area 905 or the second designated area 910 to the third designated area 1010 and selection of the third color corresponding the icons, for example, 610-9, 610-19 in the third designated area. In some embodiments, the training device 105 is also configured to transmit the training device communication signals to the third subset of wearable devices, for example, 115-9, 115-19 to illuminate the wearable device visual indicator(s), for example, 220-9, 220-19 of the third subset of wearable devices, for example, 115-9, 115-19 associated with the players, for example, 615-9, 615-19 in the third color. The illumination of the wearable device visual indicator(s), for example, 220-9, 220-19 in the third color indicates commencement of the second predefined activity corresponding to the players, for example, 615-9, 615-19 playing for the first virtual team 715 and/or the second virtual team 720 as ‘double agents’.

In some embodiments, the third color associated with icons, for example, 610-9, 610-19 representing the players, for example, 615-9, 615-19 can be same as the first color for a first time interval and same as the second color for a second time interval. The first time interval and the second time interval can be predefined time intervals, for example, the assigned time interval or the dynamically assigned time interval, or based on instructor/coach input received via the graphical time interval buttons 925. In such embodiments, the illumination of the wearable device visual indicator(s), for example, 220-9, 220-19, in the third color equivalent to the first color indicates a commencement of the players, for example, 615-9, 615-19 playing for the first virtual team 715 during the first time interval. Similarly, the illumination of the wearable device visual indicator(s), for example, 220-9, 220-19, in the third color equivalent to the second color indicates a commencement of the players, for example, 615-9, 615-19, playing for the second virtual team 720 during the second time interval of the training session associated with the first predefined activity. In some embodiments, the second predefined activity corresponds to players, for example, 615-9, 615-19, optionally playing for the first virtual team 715 or the second virtual team 720 during the training session based on a preference of the players during the second training session or the second predefined activity. In such embodiments, the third color is different from the first color and the second color.

In some embodiments, the training device 105 is configured to associate the at least one icon, for example, 610-1, 610-2 of the first subset of icons, for example, 610-1 . . . 610-10 with the third color such that a count of the first subset of icons, for example, 610-3 . . . 610-10 representing the first subset of players, for example, 615-3 . . . 615-10 associated with the first color is different from a count of the icons, for example, 610-1 . . . 610-2 representing the players associated with the third color after the association. In some embodiments, the training device 105 is also configured to associate one or more icons, for example, 610-11, 610-12 of the second subset of icons, for example, 610-11 . . . 610-20 with a fourth color such that a count of the second subset of icons, for example, 610-13 . . . 610-20 representing the second subset of players, for example, 615-13 . . . 615-20 associated with the second color is different from a count of the icons, for example, 610-11, 610-12 representing the players, for example 615-11, 615-12 associated with the fourth color after the association. In such embodiments, the training device 105 is also configured to transmit, via the training device processor 325, one or more training device communication signals to one or more wearable devices, for example, 115-1, 115-2 and 115-11, 115-12 of the plurality of wearable devices, for example, 115-1, 115-2, 115-3 . . . 115-n corresponding to the one or more players, for example, 615-1, 615-2, and 615-11, 615-12 representing the icons, for example, 610-1, 610-2, and 610-11, 610-12 associated with the third color and the fourth color respectively to illuminate the wearable device visual indicator(s), for example, 220-1, 220-2, and 220-11, 220-12 of respective wearable devices, for example, 115-1, 115-2 and 115-11, 115-12 associated with the players, for example, 615-1, 615-2, and 615-11, 615-12 in the third color and the fourth color respectively. In such embodiments, the illumination of the wearable device visual indicator(s), for example, 220-1, 220-2, and 220-11, 220-12 in the third color and the fourth color respectively indicates commencement of the third training session or the third predefined activity. In some embodiments, the third training session or the third predefined activity corresponds to a game to be played between the first subset of players, for example, 615-3 . . . 615-10 associated with the first color and the players, for example, 615-1, 615-2 associated with the third color. In some embodiments, the third training session or the third predefined activity also corresponds to a game to be played between the second subset of players, for example, 615-13 . . . 615-20 associated with the second color and the players, for example, 615-11, 615-12 associated with the fourth color.

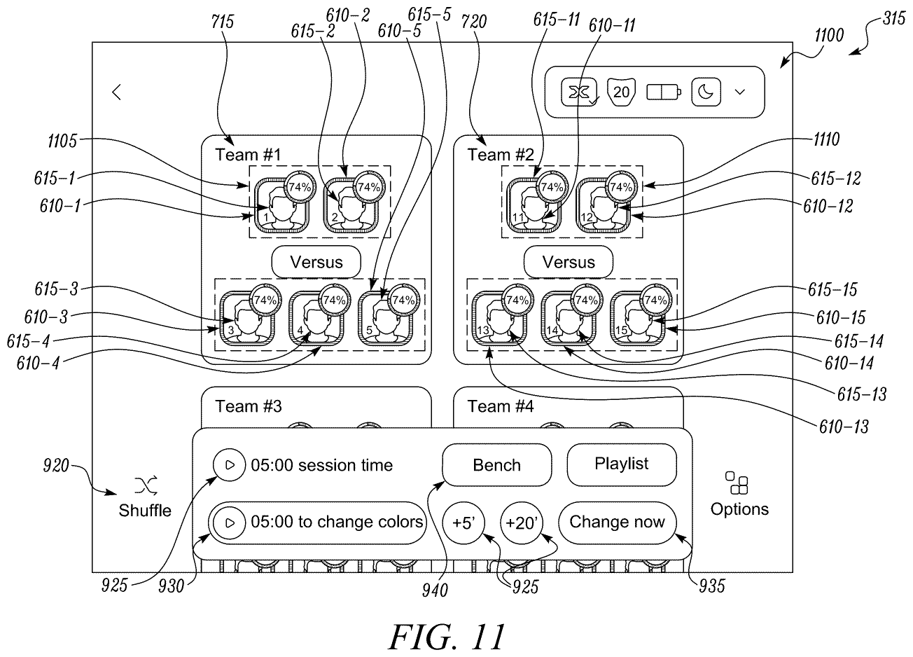

FIG. 11 illustrates an exemplary unbalanced team session user interface 1100 provided on the training device graphical user interface 315 of the training device of FIG. 3 for conducting the third training session or the third predefined activity. In some embodiments, the unbalanced team session corresponds to a sporting game, or any other activity played between at least two virtual teams of the created set of randomized virtual teams, for example, 715, 720, 725 (see FIG. 7) or at least two sets of players in each virtual team, for example, 715, 720, having different counts of players of the players, for example, 615-1 . . . 615n (see FIG. 6) in the at least two teams or the at least two sets of players respectively. For example, in some embodiments, the third training session or the third predefined activity is conducted between one or more players, for example, 615-1 . . . 615-2 of the first subset of players, for example, 615-1 . . . 615-10 correspondingly representing one or more icons, for example, 610-1 . . . 610-2 of the first subset of icons, for example, 610-1 . . . 610-10 associated with the first color and a third subset of players, for example, 1105, including the players, for example, 615-3 . . . 615-5, corresponding representing the icons, for example, 610-3, 610-5 associated with the third color. In such embodiments, the third training session or the third predefined activity is also conducted between one or more players, for example, 615-11 . . . 615-12 of the second subset of players representing the icons, for example, 610-11 . . . 610-12 associated with the second color and a fourth subset of players, for example, 1110, including the players, for example, 615-13 . . . 615-15 associated with the fourth color in the second virtual team 720.

In view of the preceding description, it will be apparent to those of ordinary skill in art that, in some embodiments, training device 105 is also configured to automatically associate a plurality of distinct colors in addition to the first color, the second color, the third color and the fourth color corresponding to an icon of the plurality of icons, for example, 610-1 . . . 610-n or groups of icons, for example, the first subset of icons, for example, 610-1 . . . 610-10 and transmit corresponding training device communication signals to the corresponding wearable devices, for example, 115-1, 115-2, 115-3 . . . 115-n of the players, representing the icons or the group of icons respectively to indicate commencement of a plurality of different predefined activities in the addition to the first predefined activity, the second predefined activity, and/or third predefined activity. It will also be appreciated by those of ordinary skill in art, in some embodiments, that training device 105 is also configured to associate the plurality of distinct colors in addition to the first color, the second color, the third color, and the fourth color corresponding to the created set of randomized virtual teams 710 (see FIG. 7) in addition to the first virtual team 715 including the first subset of players, for example, 615-1 . . . 615-10 and the second virtual team 720 including the second subset of players, for example, 615-11 . . . 615-20. In some embodiments, the training device 105 is also configured to transmit the corresponding training device communication signals to the wearable devices, for example, 115-1, 115-2, 115-3. 115-n of the players, for example, 615-1 . . . 615-n included in the additional virtual teams of the created set of randomized virtual teams 710 respectively to illuminate the wearable device visual indicator(s) 220-1, 220-2, 220-3 . . . 220-n, of the wearable devices, for example, 115-1, 115-2, 115-3 . . . 115-n respectively in the associated colors. In accordance with various embodiments, the training device 105 is also configured to transmit the corresponding training device communication signals to the wearable devices directly via the network 110 or the server 120. In accordance with various embodiments, the illumination of wearable device visual indicator(s), for example, 220-1 . . . 220-n in the associated colors indicates the commencement of the first predefined activity, the second predefined activity or a plurality of different predefined activities between or corresponding to the additional virtual teams. Further, in accordance with various embodiments, the commencement of the predefined activities based on the illuminated colors can be based on an instruction, a communication, and and/or an agreement between the instructor/coach and the players associated with the wearable devices 115-1, 115-2, 115-3 . . . 115-n.

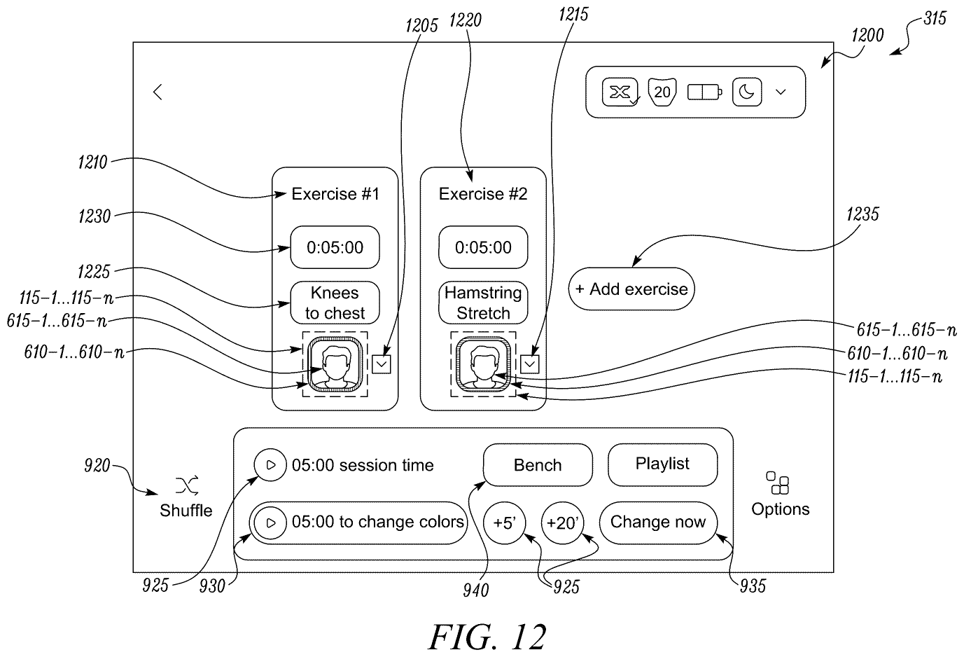

For example, FIG. 12 illustrates an exemplary warmup session user interface 1200 provided on the training device graphical user interface 315 of the training device of FIG. 3 for conducting a fourth training session or a fourth predefined activity. In some embodiments, the warmup session corresponds to an exercise session, or any other session including a series of exercises or activities to be performed by one or more players, for example, 615-1 . . . 615-n or one or more virtual teams, for example, 715, 720, 725 (see FIG. 7) consecutively. The training device 105 is configured to associate a fifth color 1205 distinct from the first color, second color, the third color, and/or the fourth color with at least one icon, for example, 610-9 (see FIG. 10), or a group of icons, such as, the first subset of icons, for example, 610-1 . . . 610-10 (see FIG. 9), the second subset of icons, for example, 610-11 . . . 610-20 (see FIG. 9), or the icons 610-1 . . . 610-n (see FIG. 6) and transmit a fifth training device communication signal to the corresponding wearable device(s), for example, 115-1 . . . 115-n associated with the players, for example, 615-1 . . . 615-n (see FIG. 6) representing the icons, for example, 610-1 . . . 610-n associated with the first color to illuminate the wearable device visual indicator(s), for example, 220-1 . . . 220-n of the wearable devices, for example, 115-1 . . . 115 —-n receiving the fifth training device communication signal in the fifth color 1205. In some embodiments, the illumination of the wearable device visual indicator(s), for example, 220-1 . . . 220-n in the fifth color 1205 indicates commencement of the fourth predefined activity by the player/players. In some embodiments, the fourth predefined activity corresponds to a first exercise 1210 to be performed by the player or the players. Similarly, the training device 105 is configured to associate a sixth color 1215 with one or more icons and transmit a sixth signal to the wearable devices, for example, 115-1, 115-2, 115-3 . . . 115-n of players, for example, 615-1. 615-n representing the icons, for example, 610-1 . . . 610-n associated with the sixth color 1215 to indicate commencement of a fifth predefined activity by the player/players. In some embodiments, the fifth predefined activity corresponds to a second exercise 1220 to be performed by the player or the players after the fourth predefined activity or the first exercise 1210. In some embodiments, the training device 105 is also configured to receive instructor/coach input, via the training device processor 325, one or more activity parameters associated with the predefined activities, via the training device graphical user interface 315. Examples of the activity parameters include, but are not limited to, activity name 1225, activity duration 1230. In some embodiments, the training device 105 is configured to change the associated color from fifth color 1205 to the sixth color 1215 at predefined time intervals, for example, the assigned time interval or the dynamically assigned time interval, automatically or based on one or more instructor/coach inputs received, via, for example, a graphical color change button 1140 the training device graphical user interface 315. In some embodiments, the training device 105 is also configured to randomly identify, select, and associate one or more icons of the plurality of icons, for example, 610-1 . . . 610-n with one or more colors at the predefined time intervals, for example, the assigned time interval or the dynamically assigned time interval, automatically or based on one or more operator/coach inputs received via the training device graphical user interface 315 or any other input means/mechanism. In some embodiments, the training device 105 is configured to automatically generate and present a sixth predefined activity after the fifth predefined activity to be performed by the players, for example, 615-1 . . . 615-n, or receive the instructor/coach input corresponding to the sixth predefined activity subsequent to the fifth predefined activity via, for example, an add activity button 1235 provided on the training device graphical user interface 315. In such embodiments, the training device 105 is configured to automatically generate the one or predefined activities based on one or more artificial intelligence models and/or the historical data including, but not limited to, previously conducted training sessions and/or predefined activities, and previous player performance evaluated corresponding to the previously conducted training sessions and/or predefined activities.

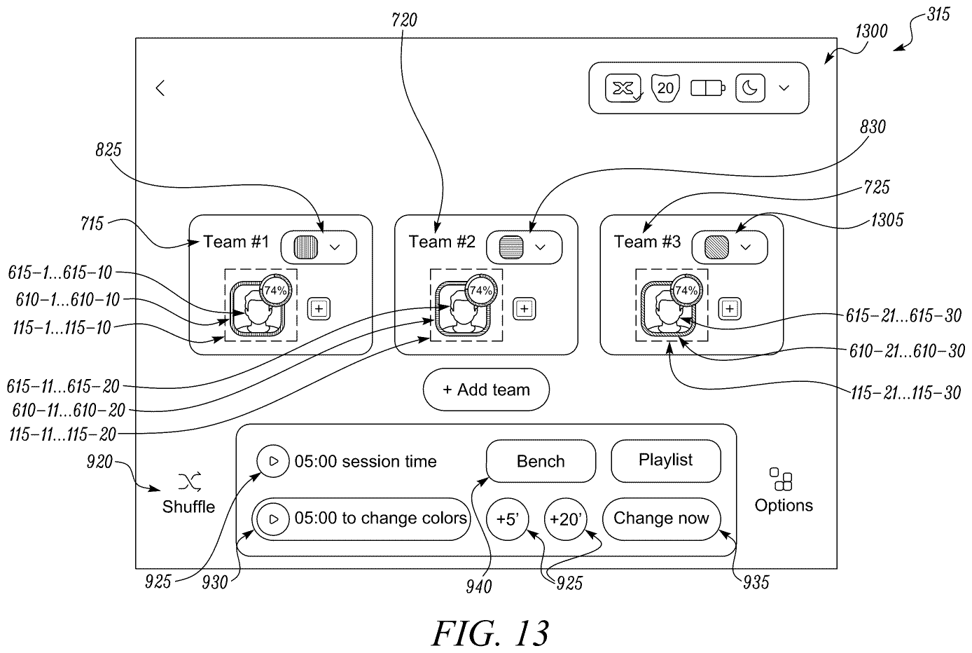

In yet another example, FIG. 13 illustrates an exemplary freeplay session user interface 1300 provided on the training device graphical user interface 315 of the training device of FIG. 3 for conducting a seventh training session or a seventh predefined activity. In some embodiments, the freeplay session corresponds to a plurality of predefined activities to be played by the plurality of players, for example, 615-1 . . . 615-n (see FIG. 6) or the created set of randomized virtual teams, for example, 715, 720, 725 (see FIG. 7) independently. The training device 105 is configured to associate a third virtual team 725 and a third subset of icons, for example, 610-21 . . . 610-30 (see FIG. 6) representing a third sub-set of players, for example, 615-21 . . . 615-30 with a seventh color 1305 and transmit the training device communication signals to the third subset of wearable devices, for example, 115-21 . . . 115-30 associated with the third sub-set of players, for example, 615-21 . . . 615-30 to illuminate the wearable device visual indicator(s), for example, 220-21 . . . 220-30 of the third subset of wearable devices, for example, 115-21 . . . 115-30 respectively in the seventh color 1305. In some embodiments, the illumination of the wearable device visual indicator(s), for example, 220-1 . . . 220-10, 220-11 . . . 220-20, and 220-21 . . . 220-30 in the first color 825, the second color 830, and the seventh color 1305 by the first subset of wearable devices, for example, 115-1 . . . 115-10, the second subset of wearable devices, for example, 115-11 . . . 115-20, and the third subset of wearable devices, for example, 115-21 . . . 115-30 respectively, indicates the commencement of the seventh predefined activity. In some embodiments, the seventh predefined activity is similar to the first predefined activity, and corresponds to, for example, a game to be played between the first subset of players, for example, 615-1 . . . 615-10, the second subset of players, for example, 615-11 . . . 615-20, and the third subset of players, for example, 615-21 . . . 615-30. In some embodiments, the illumination of the wearable device visual indicator(s), for example, 220-1 . . . 220-10, 220-11 . . . 220-20, and 220-21 . . . 220-30, in the first color 825, the second color 830, and the seventh color 1305 by the first subset of wearable devices, for example, 115-1 . . . 115-10, the second subset of wearable devices, for example, 115-11 . . . 115-20, and the third subset of wearable devices, for example, 115-21 . . . 115-30 respectively, indicates the commencement of the eighth predefined activity. In some embodiments, the eighth predefined activity is similar to the second predefined activity, and corresponds to, for example, the third subset of players, for example, 615-21 . . . 615-30 optionally playing for the first virtual team 715 or the second virtual team 720. In some embodiments, the illumination of the wearable device visual indicator(s), for example, 220-21 . . . 220-30 in the seventh color 1305 by the third subset of wearable devices, for example, 115-21 . . . 115-30 corresponds to a ninth predefined activity. In some embodiments, the ninth predefined activity is similar to the fourth predefined activity and corresponds to, for example, the third subset of players, for example, 615-21 . . . 615-30 performing the first exercise 1210 (see FIG. 12).

In some embodiments, the training device 105 is also configured to determine and/or evaluate a performance of the players, for example, 615-1 . . . 615-n (see FIG. 6) during or after the various training sessions and the predefined activities performed by the players. For example, the training device 105 is configured to determine the performance of each of the first subset of players, the second subset of players, the at least one player, or any combination thereof corresponding to the first predefined activity, the at least one subsequent activity, or a combination thereof. In some embodiments, the training device 105 is configured to determine and/or evaluate a performance based on the at least one received image, video, or a combination thereof from the image capturing device(s) 125. In some embodiments, the training device 105 is configured to receive the at least one received image, video, or a combination thereof from the image capturing device(s) 125 directly via the network 110 or via the server 120. In some embodiments, the training device 105 is also configured to determine the at least one subsequent activity to be performed by the players, for example, 615-1 . . . 615-n, for example, first subset of players, the second subset of players, the at least one player, or any combination thereof based on the determined performance during or after a current time interval of the plurality of time intervals associated with the current predefined activity. For example, training device 105 is configured to dynamically generate and/or determine the second predefined activity based on the determined performance of the players, for example, first subset of players and the second subset of players during the first predefined activity being performed.

In some embodiments, the training device 105 is configured to rank the determined performance of players, for example, the first subset of players, the second subset of players, the at least one player, or any combination thereof based on at least one performance metric corresponding to the first predefined activity, the at least one subsequent activity, or a combination thereof. Examples of the at least one performance metric include, but not limited to, an average reaction time and/or a decision quality during the predefined activities being performed, consistency patterns in movement, behavior, and/or actions, speed of movement during the predefined activities of varying intensities, and temporal evolution or improvement in performance over multiple predefined activities of each player. The average reaction time corresponds to a time taken by a player, for example, 615-1 to respond to a stimulus such as, but not limited to, a receiving the playing ball from another player. The decision quality determines an effectiveness of the decision(s) taken by the player during the predefined activities being performed. In some embodiments, the training device 105 is configured to evaluate the performance metric associated with the temporal evolution or improvement in performance based on the at least one received image and/or video corresponding to different predefined activities and/or different timelines including, but not limited to, the predefined activities performed in a day, during a week, and/or a month.

In some embodiments, the training device 105 is configured to implement at least one machine or computer vision analysis methods, algorithms, and/or models and/or the at least one artificial intelligence model, corresponding to the at least one received image and/or video from the image capturing device(s) 125 to automatically evaluate and/or determine performance of each player of the players, for example, 615-1 . . . 615-n (see FIG. 6) based on the at least one performance metric. In some embodiments, the training device 105 is configured to determine various aspects and/or events associated with each player and/or the predefined activities being performed based on the machine vision analysis. For example, the training device 105 is configured to track the players, for example, 615-1 . . . 615-n, performing the predefined activities in a playing field based on one or more features, including, but not limited to, a color, a number, a design, and/or pattern, associated with the wearable device, for example, 115-1 (see FIG. 1) and/or a garment such as a jersey adorned by each player. Similarly, the training device 105 is also configured to determine, via the machine vision analysis, the events including, but not limited to, a playing ball reception by each player, a passing of the playing ball made between the players, change of direction of each player and/or the playing ball, and/or a loss or recovery of each player during the predefined activities. In some embodiments, the training device 105 is also configured to determine, via the machine vision analysis, timestamps and one or more cognitive reaction markers of each player associated with each determined event during the predefined activities being performed. Examples of the cognitive reaction markers include, but are not limited to, a time taken by a player to decide an action after receiving the playing ball, another player chosen by the player to pass the playing ball among different players in proximity to the player, and/or decisions taken by the player during predefined activities being performed based on one or more situations or tactical scenarios encountered by the player during the predefined activities. In accordance with various embodiments, the training device 105 is also configured to determine the performance of the players, for example, 615-1 . . . 615-n based on the at least one received input from the wearable device sensor(s) 230-1 of the wearable devices, for example, 115-1 . . . 115-n associated with the players.

In some embodiments, the training device 105 is also configured to rank the determined performance of each player, for example, the first subset of players, the second subset of players, the at least one player, or any combination thereof based on at least one performance metric corresponding to the different predefined activities, for example, first predefined activity, the at least one subsequent activity, or a combination thereof, performed or being performed. In some embodiments, the training device 105 is also configured to compare the determined performance of the players, for example, 615-1 . . . 615-n (see FIG. 6) with respect to each other corresponding to the one or more predefined activities performed or being performed based in the at least one determine performance metric corresponding to each player. As an example, the training device 105 is also configured to compare the determined performance of first subset of players, the second subset of players, the at least one player, or any combination thereof with respect to each other corresponding to the first predefined activity, the at least one subsequent activity, or a combination thereof. In some embodiments, the training device 105 is also configured to provide at least recommendation associated with each player, for example, the first subset of players, the second subset of players, the at least one player, or any combination thereof based on the determined performance, the ranking, the comparison, or a combination thereof corresponding to each predefined activity, for example, the first predefined activity, the at least one subsequent activity, or a combination thereof. Examples of the at least recommendation includes, but is not limited to, recommendations associated with team formation, strategy, and/or placement of the players, for example, 615-1 . . . 615-n, during a game, change or improvement in the generated predefined activities, areas of improvement for each player, and/or change or improvement in player skills. In some embodiments, the training device 105 is also configured to automatically perform one or more actions based on the determined performance of the players, for example, 615-1 . . . 615n. For example, the training device 105 is configured to automatically adjust, modify, and/or change the previously generated predefined activities corresponding to one or more players based on the determined performance, and automatically implement the adjusted, modified, and/or changed predefined activities for subsequent training sessions of the players, for example, 615-1 . . . 615-n (see FIG. 6). In some embodiments, the change, adjustment, and/or the modification of the predefined activities correspond to modifying the predefined activity and/or modifying a position, placement, role/responsibility of the one or more players for the subsequent training sessions. In some embodiments, the training device 105 is also configured to create one or more video clips based on the at least one received image and/or video from the image capturing device(s) 125 corresponding to the detected and/or determined events. In some embodiments, the training device 105 is also configured to provide the one or more created video clips along the at least one recommendation for review and analysis by the players, for example, 615-1 . . . 615-n and/or the instructor/coach.

In some embodiments, the training device 105 is also configured to enable the instructor/coach to evaluate performance of the players, for example, 615-1 . . . 615-n (see FIG. 6) during or after the various training sessions and the predefined activities performed by the players based on a visual evaluation conducted by the instructor/coach. In some embodiments, the training device 105 is also configured to enable the instructor/coach to assign scores corresponding to the players via the training device graphical user interface 315 or any other input means/mechanism. In such embodiments, the training device 105 is also configured to receive the instructor/coach input related to the scores and display the scores corresponding to the players via, for example, a leaderboard interface (not shown) provided on the training device graphical user interface 315. In some embodiments, the training device 105 is configured to display the leaderboard interface including a list of the players and corresponding scores associated with the players in a descending order of the scores. In some embodiments, the training device 105 is also configured to automatically generate and display the scores corresponding to the players, for example, 615-1 . . . 615-n via the training device graphical user interface 315 based on the evaluation of the players, for example, 615-1 . . . 615-n by the instructor/coach. In some embodiments, the training device 105 is configured to implement one or more artificial intelligence models or predefined evaluation scoring models to generate the scores.

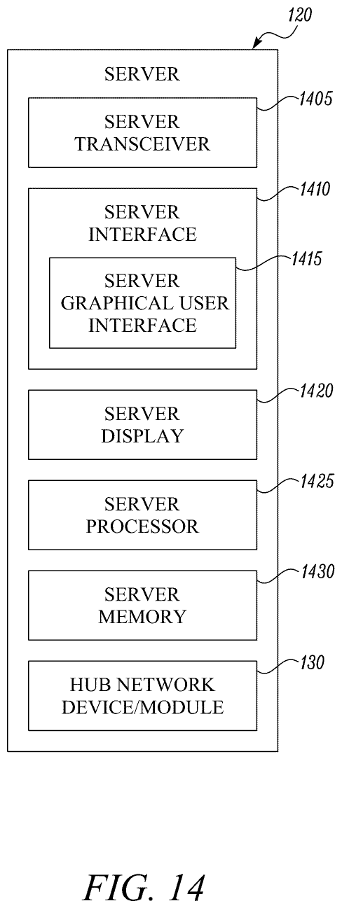

It also contemplated that server 120 of FIG. 1 is also configured to perform one or more functions of the training device 105 as outlined in the present disclosure with respect to FIG. 6 through FIG. 13. The various components of server 120 will now be described hereinafter with respect to FIG. 14. It should be appreciated by those of ordinary skill in the art that FIG. 14 depicts the server 120 in a simplified manner and a practical embodiment includes additional components and suitably configured logic to support known or conventional operating features that are not described in detail herein. Although the components of the server 120 are illustrated and described to be implemented within the server 120 it is contemplated that the one or more components of the server 120 can alternatively be implemented in a distributed computing environment and/or implemented to be in remote and/or retrofitted communication with the server 120.

Referring to FIG. 14, a block diagram of the exemplary server 120 of FIG. 1 in communication with the training device 105 (see FIG. 3) and the wearable devices, for example, 115-1 . . . 115-n (see FIG. 1) via the network 110 (see FIG. 1) is disclosed. It will be understood with those with ordinary skill in the art that the server 120 is configured to include similar components as the training device components, for example, 305 through 330, 130 and configured to perform similar functions as the training device components, for example, 305 through 330, 130. For example, the server 120 includes the server transceiver 1405, server interface 1410, server graphical user interface 1415, server display 1420, server processor 1425, server memory 1430, and the hub network device/module 130 of the server. In some embodiments, the server 120 is configured to communicate with and facilitate the communication between the training device 105 (see FIG. 3) and the wearable devices, for example, 115-1 . . . 115-n via the server transceiver 1405. In some embodiments, the server memory 1430 is configured to store at least one server information associated with the training device 105 and/or the wearable devices, for example, 115-1, 115-2, 115-3 . . . 115-n. Examples of the server information include, but are not limited to, wearable device identification of each wearable device of the wearable devices, for example, 115-1, 115-2, 115-3 . . . 115-n, a player associated with a corresponding wearable device, for example, 115-1, a previous pairing or association between the training device 105 and one or more of the wearable devices, for example, 115-1, 115-2, 115-3 . . . 115-n, one or more training sessions conducted using the training device 105 and the wearable devices, for example, 115-1, 115-2, 115-3 . . . 115-n, data associated with the one or more training sessions conducted, and one or more images and/or video content associated with the training sessions and/or the players captured by the image capturing device(s) 125. In some embodiments, the server 120 is configured to receive the at least one server information from the training device 105 and/or the wearable devices, for example, 115-1, 115-2, 115-3 . . . 115-n via the network 110 and one or more network devices including, but not limited to, one or more routers. In some embodiments, the server 120 is configured to automatically create the randomized set of virtual teams 710 (see FIG. 7) of the players, for example, 615-1 . . . 615-n (see FIG. 6), generate the one or more predefined activities to be performed by one or more of the created set of randomized virtual teams 710 at different time intervals, and commence the one or more generated predefined activities by associating and illuminating one or more visual indicators of the wearable devices, for example, 115-1 . . . 115-n (see FIG. 1) in one or more colors similar to the training device 105 as shown in FIGS. 6 through 13. In some embodiments, the server 120 is configured to provide the created set of randomized teams 710, the generated predefined activities to the training device 105 via the network 110. In some embodiments, the server 120 is also configured to commence and coordinate the predefined activities to be performed and/or being performed directly with the wearable devices, for example, 115-1 . . . 115-n via the network 110 and also provide information including, but not limited to, the commencement of the predefined activities, the association of colors corresponding to the wearable devices and/or the time intervals or the change in the time intervals associated with the predefined activities to be performed and/or being performed to the training device 105 via the network 110. In some embodiments, the server 120 is also configured to determine the performance of the players, for example, 615-1 . . . 615-n (see FIG. 6) based on the one or more images and/or video content associated with the training sessions and/or the players captured by the image capturing device(s) 125 similar to the training device 105. In some embodiments, the server 120 is also configured to generate the at least one recommendation corresponding to one or more players and/or the predefined activities based on the determined performance. In some embodiments, the server 120 is also configured to provide the determined performance and the at least one recommendation to the training device 105 via the network.

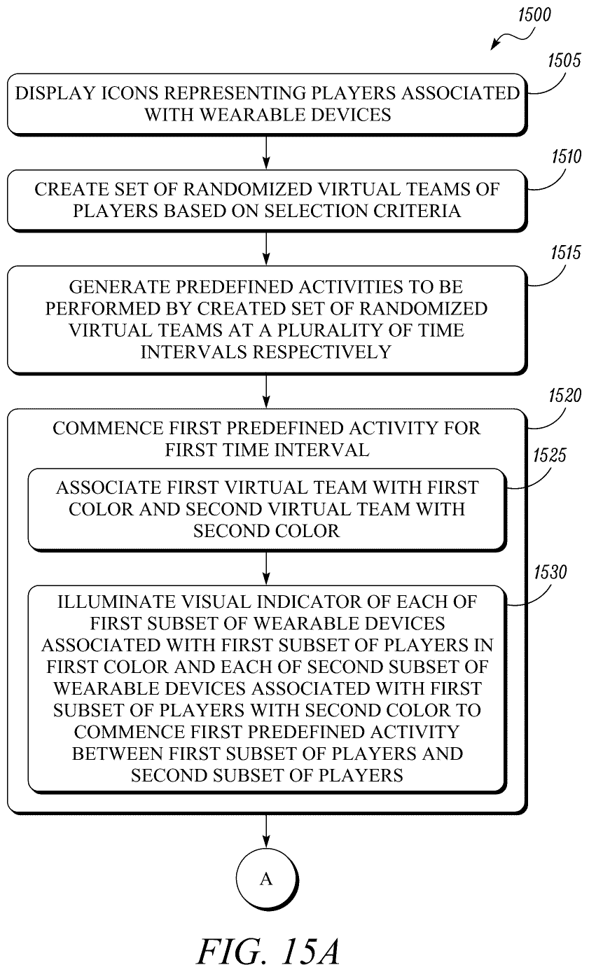

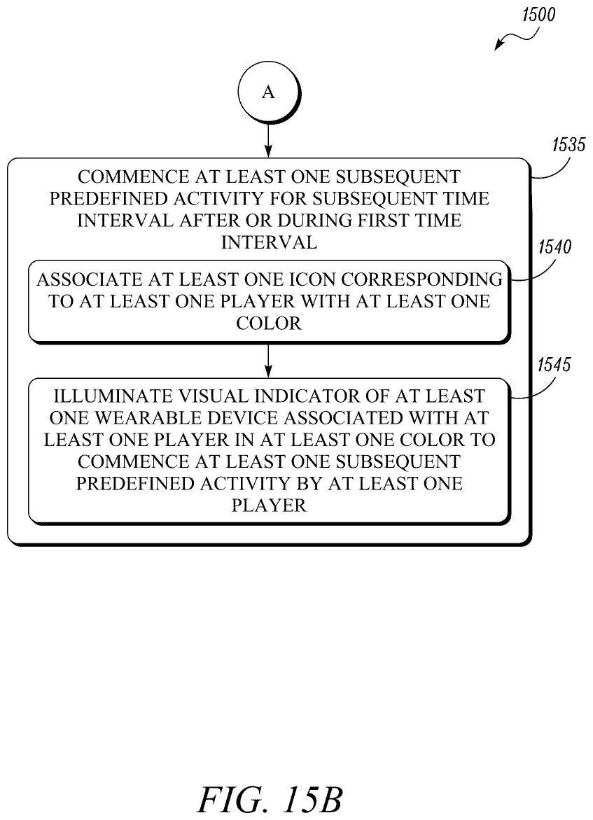

FIGS. 15A through 15B illustrate an exemplary method 1500 for conducting a training session for a plurality of players using the training device 105 of FIG. 3. At 1505, the training device 105 displays, via the training device graphical user interface 315, the plurality of icons, for example, 610-1 . . . 610-n (see FIG. 6) correspondingly representing the players, for example, 615-1 . . . 615-n (see FIG. 6) associated with a plurality of wearable devices, for example, 115-1, 115-2, 115-3 . . . 115-n (see FIG. 6). At 1510, the training device 105 automatically creates, via the training device processor and the at least one artificial intelligence model implemented by the training device processor 325 (see FIG. 3), the created set of randomized virtual teams 710 (see FIG. 7). At 1510, the training device 105 creates, via the training device processor 325, the first virtual team 715 on the training device graphical user interface 315 by selecting the first subset of icons, for example, 610-1 . . . 610-10 of the plurality of icons, for example, 610-1 . . . 610-n corresponding to the first subset of players, for example, 615-1 . . . 615-10 of the plurality of players, for example, 615-1 . . . 615-n. At 1510, the training device 105 also creates, via the training device processor 325, a second virtual team 720 on the training device graphical user interface 315 based selecting the second subset of icons, for example, 610-11 . . . 610-20 of the plurality of icons, for example, 610-1 . . . 610-n corresponding to the second subset of players, for example, 615-11 . . . 615-20 of the plurality of players, for example, 615-1 . . . 615-n. The first subset of players is different from the second subset of players. At 1515, the training device 105 automatically generates, via the training device processor 325 and the at least one artificial intelligence model, a plurality of predefined activities to be performed by the created set of randomized virtual teams 710 at a plurality of time intervals respectively. At 1520, the training device 105 automatically commences, via the training device processor 325 and the at least one artificial intelligence model, a first predefined activity of the plurality of predefined activities for a first time interval of the plurality of time intervals. At 1525, the training device 105 associates, via the training device processor 325, the first virtual team 715 and the first subset of icons, for example, 610-1 . . . 610-10 with the first color 825 (see FIG. 8) and the second virtual team 720 and the second subset of icons, for example, 610-11 . . . 610-20 with the second color 830 (see FIG. 8). At operation 1530, the training device 105 illuminates the wearable device visual indicator(s), for example, 220-1 . . . 220-10 of each of the first subset of wearable devices, for example, 115-1 . . . 115-10 in the first color 825the wearable device visual indicator(s), for example, 220-11 . . . 220-20 of each of the second subset of wearable devices, for example, 115-11 . . . 115-20 in the second color 830 to commence the first predefined activity. At 1535, the training device 105 commences at least one subsequent predefined activity of the plurality of predefined activities that is different from the first predefined activity for a subsequent time interval of the plurality of time intervals after or during the first time interval. At 1540, the training device 105 associates, via the training device processor 325, at least one icon, for example, 610-9, 610-19 of the plurality of icons, for example, 610-1 . . . 610-n corresponding to at least one player, for example, 615-9 with at least one color of the plurality of distinct colors. At 1545, the training device 105 illuminates the wearable device visual indicator(s), for example, 220-9, 220-19 of the wearable devices, for example, 115-9, 115-19 in the at least one color to commence the at least one subsequent predefined activity during or after the first predefined activity.

In view of the foregoing, it will be apparent that the system 100, the training device 105, and method 1500 of the present disclosure, provide an interactive and effective solution to an instructor or a coach for conducting engaging training sessions for the players by involving the players in different teams, different predefined activities, and exercise routines at different time intervals based on visual indicators in real-time. Such various of the training sessions or activities, enable the instructor/coach to improve coordination and agility of the players. The present disclosure also allows the instructors to control the time durations of sessions, introduce activity changes during sessions, and control rest times for the players during or after the training sessions. The present disclosure also enables the instructor or the coach to conduct such training sessions at any given point in time with minimal input by implementing machine learning and/or artificial intelligence models and/or algorithms to automatically conduct the training sessions based on previously conducted training sessions by the instructor/coach. The use of artificial intelligence models also allows the instructor/coach to focus on supervision and evaluation of performance of the players during such sessions rather than manually conducting the sessions each time. Moreover, use of the visual indicators as described in the present disclosure, allows the instructor/coach to visually identify each player and the respective activities being performed by each player during the training sessions and also avoids needless and repeated verbal instructions to be provided to the players to perform the different activities during the training sessions.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that can cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover, in this document, relational terms such as first and second, top and bottom, front and rear, and the like can be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but can include other elements not expressly listed or inherent to such process, method, article, or apparatus. An element preceded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way but can also be configured in ways that are not listed.

It will be appreciated that some embodiments can be comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A system for conducting a training session for a plurality of players, the system comprising:
a plurality of wearable devices correspondingly associated with the plurality of players, wherein each wearable device of the plurality of wearable devices comprises a visual indicator capable of being illuminated in a plurality of distinct colors; and
a training device associated with an instructor and in communication with the plurality of wearable devices, wherein the training device is configured to automatically:
display, via a user interface, a plurality of icons correspondingly representing the plurality of players associated with the plurality of wearable devices in communication with the training device;
create, via a processor of the training device and at least one artificial intelligence model implemented by the processor, a set of randomized virtual teams of the plurality of players on the user interface based on a count of the plurality of wearable devices in communication with the training device, wherein the creating of a first virtual team of the created set of randomized virtual teams comprises selecting a first subset of icons of the plurality of icons corresponding to a first subset of players of the plurality of players, and the creating of a second virtual team of the created set of randomized virtual teams comprises selecting a second subset of icons of the plurality of icons corresponding to a second subset of players of the plurality of players, and wherein the first subset of players is different from the second subset of players; create, via a processor of the training device and at least one artificial intelligence model implemented by the processor, a set of randomized virtual teams of the plurality of players on the user interface based a count of the plurality of wearable devices in communication with the training device, wherein the creating of a first virtual team of the created set of randomized virtual teams comprises selecting a first subset of icons of the plurality of icons corresponding to a first subset of players of the plurality of players, and the creating of a second virtual team of the created set of randomized virtual teams comprises selecting a second subset of icons of the plurality of icons corresponding to a second subset of players of the plurality of players, and wherein the first subset of players is different from the second subset of players;
generate, via the processor and the at least one artificial intelligence model, a plurality of predefined activities to be performed by the created set of randomized virtual teams at a plurality of time intervals respectively;
commence, via the processor and the at least one artificial intelligence model, a first predefined activity of the plurality of predefined activities for a first time interval of the plurality of time intervals by:
associating, via the processor, the first virtual team with a first color of the plurality of distinct colors and the second virtual team with a second color of the plurality of distinct colors, and
illuminating, via the processor and a transceiver of the training device, the visual indicator of each of a first subset of wearable devices of the plurality of wearable devices in the first color and the visual indicator of each of a second subset of wearable devices of the plurality of wearable devices associated with the second subset of players with the second color, wherein the illumination of the visual indicator in the first color and the second color is indicative of the commencement of the first predefined activity between the first subset of players and the second subset of players; and
commence, via the processor and the at least one artificial intelligence model, at least one subsequent predefined activity of the plurality of predefined activities that is different from the first predefined activity for a subsequent time interval of the plurality of time intervals after or during the first time interval by:
associating, via the processor and the at least one artificial intelligence model, at least one icon of the plurality of icons corresponding to at least one player of the plurality of players with at least one color of the plurality of distinct colors; and
illuminating, via the processor and the transceiver, the visual indicator of at least one wearable device of the plurality of wearable devices in the at least one color, wherein the illumination of the visual indicator in the at least one color is indicative of the commencement of the at least one subsequent predefined activity by the at least one player.

2. The system of claim 1, wherein the training device is configured to:
transmit, via the processor and the transceiver, a first signal to the first subset of wearable devices corresponding to the first subset of players and a second signal to the second subset of wearable devices corresponding to the second subset of players, wherein the first and second subset of wearable devices are configured to illuminate the visual indicator in the first color and the second color in response to the received first signal and the received second signal respectively; and
transmit, by the processor and the transceiver, a third signal to the at least one wearable device corresponding to the at least one player, wherein the at least one wearable device is configured to illuminate the visual indicator in the at least one color in response to the received third signal.

3. The system of claim 1, wherein the training device is configured to create the set of randomized virtual teams based on at least one selection criteria, wherein the at least one selection criteria comprises:
at least one historical performance of the plurality of players,
at least one historical virtual team of the plurality of players,
at least one skill associated with the plurality of players,
at least one predefined team composition criteria defining the at least one skill, the
at least one historical performance, at least one complimentary skill of the at least one skill between the plurality of players, or any combination thereof corresponding to the set of randomized teams to be created,
at least one predetermined strength, weakness, or a combination thereof of each player of the plurality of players,
a randomized selection of the plurality of players, or
any combination thereof.

4. The system of claim 1, wherein the generating of the plurality of predefined activities comprises:
generating via the processor and the at least one artificial intelligence model, a randomized sequence of the plurality of predefined activities to be performed in a sequence prior to the commencement of the first predefined activity, or
generating, via the processor and the at least one artificial intelligence model, the first predefined activity; and
dynamically generating, via the processor and the at least one artificial intelligence model, each subsequent predefined activity of the plurality of predefined activities to be performed after the first predefined activity in real-time, wherein each subsequent predefined activity is dynamically generated prior to, during, or after the commencement of a current predefined activity of the plurality of predefined activities.

5. The system of claim 1, wherein the training device is configured to:
assign, via the processor and the at least one artificial intelligence model, the plurality of time intervals corresponding to the plurality of predefined activities prior to the commencement of the first predefined activity, wherein the plurality of assigned time intervals is same or different from each other; or
dynamically assign, via the processor and the at least one artificial intelligence model, a time interval of the plurality of time intervals corresponding to each predefined activity of the plurality of predefined activities prior to the commencement of each predefined activity in real-time, wherein the dynamically assigned time interval corresponding to each predefined activity is same or different from each other.

6. The system of claim 5, wherein the training device is configured to:
dynamically modify, via the processor and the at least one artificial intelligence model, the plurality of assigned time intervals or the dynamically assigned time interval during the current predefined activity being performed.

7. The system of claim 1, wherein the at least one subsequent predefined activity corresponds to a dynamic modification of the first predefined activity including at least one additional rule applied to the first predefined activity, a change in frequency of the first predefined activity, a change in an intensity of the first predefined activity, at least one external stimuli or a change in the at least one external stimuli provided to the first subset of players, the second subset of players, the at least one player, or any combination thereof during or after the first interval of the first predefined activity, or any combination thereof.

8. The system of claim 1, wherein each wearable device comprises at least one sensor configured to provide at least sensor input associated with each of the first subset of players, the second subset of players, or a combination thereof to the training device during the first predefined activity, the at least one subsequent activity, or a combination thereof, and the system further comprises:
at least one image capturing device in communication with the training device, wherein the at least one image capturing device is configured to:
capture at least one image, video, or a combination thereof of the first subset of players, the second subset of players, or a combination thereof during the first predefined activity, the at least one subsequent activity, or a combination thereof; and
provide the at least one captured image, video, or a combination thereof to the training device

9. The system of claim 8, wherein upon receipt of the at least one captured image, video, or the combination thereof, the at least one sensor input, or a combination thereof the training device is configured to:
determine a performance of each of the first subset of players, the second subset of players, the at least one player, or any combination thereof corresponding to the first predefined activity, the at least one subsequent activity, or a combination thereof;
rank the determined performance of the first subset of players, the second subset of players, the at least one player, or any combination thereof based on at least one performance metric corresponding to the first predefined activity, the at least one subsequent activity, or a combination thereof;
compare the determined performance of the first subset of players, the second subset of players, the at least one player, or any combination thereof with respect to each other corresponding to the first predefined activity, the at least one subsequent activity, or a combination thereof;
provide at least recommendation associated with each of the first subset of players, the second subset of players, the at least one player, or any combination thereof based on the determined performance, the ranking, the comparison, or a combination thereof corresponding to the first predefined activity, the at least one subsequent activity, or a combination thereof; or
any combination thereof.

10. The system of claim 8, wherein upon receipt of the at least one captured image, video, or the combination thereof, the at least one sensor input, or a combination thereof the training device is configured to:
determine a performance of each of the first subset of players, the second subset of players, the at least one player, or any combination thereof corresponding to a current predefined activity being performed;
determine the at least one subsequent activity to be performed by first subset of players, the second subset of players, the at least one player, or any combination thereof based on the determined performance during or after a current time interval of the plurality of time intervals associated with the current predefined activity.

11. The system of claim 1, wherein each wearable device comprises a Quick-Response (QR) code and the training device is configured to establish the communication with each wearable device based on the QR code.

12. The system of claim 1, wherein the training device is configured to assign an identification of the at least one player with a corresponding wearable device of the plurality of wearable devices upon the establishment of the communication with the corresponding wearable device of the at least one player, or the training device is configured to determine the identification of at least one player with the corresponding wearable device based on at least one previous assignment of the at least one player with the corresponding wearable device.

13. The system of claim 1, wherein the first predefined activity corresponds to a game to be played between the first subset of players and the second subset of players.

14. The system of claim 1, wherein the at least one color corresponds to a third color that is same as the first color or the second color, and different from a previously color of the at least one icon corresponding to the at least one player, and further wherein associating the least one icon corresponding to the at least one player with the third color corresponds to associating the at least one icon with the first subset of icons corresponding to the first subset of players, or the second subset of icons corresponding to the second subset of players, or wherein the third color is different from the first color and the second color, and further wherein associating the least one icon corresponding to the at least one player with the third color corresponds to associating one or more of the first subset of icons or the second subset of icons with the third color.

15. The system of claim 8, wherein the training device is configured to associate the at least one icon with the third color such that a count of the first subset of icons representing the first subset of players is different from a count of the second subset of icons representing the second subset of players after the association.

16. The system of claim 1, wherein the training device is configured to:
create the first virtual team and the second virtual team by moving the corresponding first subset of icons and the second subset of icons to a first designated area and a second designated area on the user interface respectively;
associate the at least one icon of the plurality of icons with the first subset of icons or the second subset of icons comprises moving the at least one icon between the first designated area and the second designated area, wherein the associating of the at least one icon with the first subset of icons or the second subset of icons comprises: associating the at least one icon with the first color associated with the first subset of icons or the second color associated with the second subset of icons;
or perform a combination thereof.

17. The system of claim 1, wherein the at least one subsequent predefined activity corresponds to the at least one player playing for the first virtual team during a first time interval and the second virtual team during the subsequent time interval.

18. The system of claim 1, wherein the at least one artificial intelligence model is trained based on at least one previously conducted training session.

19. The system of claim 1, wherein the training device is configured to randomly select the at least one icon from the plurality of icons and associate the at least one icon with the at least one color after a predefined period of time from the commencement of the first predefined activity.

20. A method for conducting a training session for a plurality of players, the method comprising automatically:
displaying, via a user interface of a training device, a plurality of icons correspondingly representing the plurality of players associated with a plurality of wearable devices in communication with the training device, wherein each wearable device of the plurality of wearable devices comprises a visual indicator capable of being illuminated in a plurality of distinct colors;
creating, via a processor of the training device and at least one artificial intelligence model implemented by the processor, a set of randomized virtual teams of the plurality of players on the user interface based on a count of the plurality of wearable devices in communication with the training device, wherein the creating of a first virtual team of the created set of randomized virtual teams comprises selecting a first subset of icons of the plurality of icons corresponding to a first subset of players of the plurality of players, and the creating of a second virtual team of the created set of randomized virtual teams comprises selecting a second subset of icons of the plurality of icons corresponding to a second subset of players of the plurality of players, and wherein the first subset of players is different from the second subset of players;
generating, via the processor and the at least one artificial intelligence model, a plurality of predefined activities to be performed by the created set of randomized virtual teams at a plurality of time intervals respectively;
commencing, via the processor and the at least one artificial intelligence model, a first predefined activity of the plurality of predefined activities for a first time interval of the plurality of time intervals, wherein the commencing comprises:
associating, via the processor, the first virtual team with a first color of the plurality of distinct colors and the second virtual team with a second color of the plurality of distinct colors, and
illuminating, via the processor and a transceiver of the training device, the visual indicator of each of a first subset of wearable devices of the plurality of wearable devices associated with the first subset of players in the first color and the visual indicator of each of a second subset of wearable devices of the plurality of wearable devices associated with the second subset of players with the second color, wherein the illumination of the visual indicator in the first color and the second color is indicative of the commencement of the first predefined activity between the first subset of players and the second subset of players; and
commencing, via the processor and the at least one artificial intelligence model, at least one subsequent predefined activity of the plurality of predefined activities that is different from the first predefined activity for a subsequent time interval of the plurality of time intervals after or during the first time interval, wherein the commencing comprises:
associating, via the processor and the at least one artificial intelligence model, at least one icon of the plurality of icons corresponding to at least one player of the plurality of players with at least one color of the plurality of distinct colors; and
illuminating, via the processor and the transceiver, the visual indicator of at least one wearable device of the plurality of wearable devices in the at least one color, wherein the illumination of the visual indicator in the at least one color is indicative of the commencement of the at least one subsequent predefined activity by the at least one player.

21. A training device for conducting a training session for a plurality of players, the training device comprising:
a transceiver in communication with a plurality of wearable devices correspondingly associated with the plurality of players, wherein each wearable device of the plurality of wearable devices comprises a visual indicator capable of being illuminated in a plurality of distinct colors;
a processor; and
a memory for storing instructions, that when executed by the processor, causes the processor to automatically:
display, via a user interface, a plurality of icons correspondingly representing the plurality of players associated with the plurality of wearable devices in communication with the training device;
create, via at least one artificial intelligence model implemented by the processor, a set of randomized virtual teams of the plurality of players on the user interface based on a count of the plurality of wearable devices in communication with the training device, wherein the creating of a first virtual team of the created set of randomized virtual teams comprises selecting a first subset of icons of the plurality of icons corresponding to a first subset of players of the plurality of players, and the creating of a second virtual team of the created set of randomized virtual teams comprises selecting a second subset of icons of the plurality of icons corresponding to a second subset of players of the plurality of players, and wherein the first subset of players is different from the second subset of players;
generate, via the at least one artificial intelligence model, a plurality of predefined activities to be performed by the created set of randomized virtual teams at a plurality of time intervals respectively;
commence, via the at least one artificial intelligence model, a first predefined activity of the plurality of predefined activities for a first time interval of the plurality of time intervals by:
associating the first virtual team with a first color of the plurality of distinct colors and the second virtual team with a second color of the plurality of distinct colors, and
illuminating, via the transceiver, the visual indicator of each of a first subset of wearable devices of the plurality of wearable devices in the first color and the visual indicator of each of a second subset of wearable devices of the plurality of wearable devices associated with the second subset of players with the second color, wherein the illumination of the visual indicator in the first color and the second color is indicative of the commencement of the first predefined activity between the first subset of players and the second subset of players; and
commence, via the at least one artificial intelligence model, at least one subsequent predefined activity of the plurality of predefined activities that is different from the first predefined activity for a subsequent time interval of the plurality of time intervals after or during the first time interval by:
associating, via the at least one artificial intelligence model, at least one icon of the plurality of icons corresponding to at least one player of the plurality of players with at least one color of the plurality of distinct colors; and
illuminating, via the transceiver, the visual indicator of at least one wearable device of the plurality of wearable devices in the at least one color, wherein the illumination of the visual indicator in the at least one color is indicative of the commencement of the at least one subsequent predefined activity by the at least one player.
